SEQUENCE LISTING (10 sequences)

SEQ ID NO: 1 - DNA, 25 bp, Artificial Sequence
  primer
gatgtatctatctggaactctagtg

SEQ ID NO: 2 - DNA, 32 bp, Artificial Sequence
  primer
tggtaaatcatgtaaatagtaataatcaagcc

SEQ ID NO: 3 - DNA, 1467 bp, Streptococcus agalactiae
atattggtaagaagaaatttccttaaaaataagattaaataggttgtaaagtatccgtatgggttttacttgaaaaactaaattaaattatcaagaaattaccccccaggataggcgccaagaatattatacccacttgataatggtaagttttatgctaaaaatgcagtttacttgtaataatgttaaatatagggggaaagaaagcgctttgacgaccttttggacaagtagtaagataccaacatgggccctgtaaattaaaaatactgcagtagaagtgattttagtttaaaggaggaaatttattatgaacgttacacatatgatgtatctatctggaactctagtggctggtgcattgttattttcaccagctgtattagaagtacatgctgatcaagtgacaactccacaagtggtaaatcatgtaaatagtaataatcaagcccagcaaatggctcaaaagcttgatcaagatagcattcagttgagaaatatcaaagataatgttcagggaacagattatgaaaaaccggttaatgaggctattactagcgtggaaaaattaaagacttcattgcgtgccaaccctgagacagtttatgatttgaattctattggtagtcgtgtagaagccttaacagatgtgattgaagcaatcactttttcaactcaacatttaacaaataaggttagtcaagcaaatattgatatgggatttgggataactaagctagttattcgcattttagatccatttgcttcagttgattcaattaaagctcaagttaacgatgtaaaggcattagaacaaaaagttttaacttatcctgatttaaaaccaactgatagagctaccatctatacaaaatcaaaacttgataaggaaatctggaatacacgctttactagagataaaaaagtacttaacgtcaaagaatttaaagtttacaatactttaaataaagcaatcacacatgctgttggagttcagttgaatccaaatgttacggtacaacaagttgatcaagagattgtaacattacaagcagcacttcaaacagcattaaaataatatttgtatttttcgtgtgatgctgtcgacttcgtgattttgtactaccatgattgttatgattaaaagatttacgacaatagtcataatagtagaacgatgtcaccattttaaataataaagtgattagtcatttgactaaatttgccaagtatcaaaggaaataaagattatgactaaaaagataactgttgtagcattagaaacattgattgcccagcataataatatccatttgatagacgttcgtgaagagcatgagtatcgtggagggcatattccaggtgcgataaatcttcctttgagtcactcagtcataagtttgaacagttagataaaataaggaatattatcttgttggcaacgagggggaagatctattagagcat

SEQ ID NO: 4 - DNA, 124 bp, Streptococcus agalactiae
gatgtatctatctggaactctagtggctggtgcattgttattttcaccagctgtattagaagtacatgctgatcaagtgacaactccacaagtggtaaatcatgtaaatagtaataatcaagcc

SEQ ID NO: 5 - DNA, 33 bp, Artificial Sequence
  probe
agaagtacatgctgatcaagtgacaactccaca

SEQ ID NO: 6 - DNA, 21 bp, Artificial Sequence
  primer
gtgcatcaccagagcatgtat

SEQ ID NO: 7 - DNA, 26 bp, Artificial Sequence
  primer
cagctacagacagtaagttacaagcg

SEQ ID NO: 8 - DNA, 1301 bp, Streptococcus agalactiae
atgaaaatgaataaaaaggtactattgacatcgacaatggcagcttcgctattatcagtcgcaagtgttcaagcacaagaaacagatacgacgtggacagcacgtactgtttcagaggtaaaggctgatttggtaaagcaagacaataaatcatcatatactgtgaaatatggtgatacactaagcgttatttcagaagcaatgtcaattgatatgaatgtcttagcaaaaattaataacattgcagatatcaatcttatttatcctgagacaacactgacagtaacttacgatcagaagagtcatactgccacttcaatgaaaatagaaacaccagcaacaaatgctgctggtcaaacaacagctactgtggatttgaaaaccaatcaagtttctgttgcagaccaaaaagtttctctcaatacaatttcggaaggtatgacaccagaagcagcaacaacgattgtttcgccaatgaagacatattcttctgcgccagctttgaaatcaaaagaagtattagcacaagagcaagctgttagtcaagcagcagctaatgaacaggtatcaacagctcctgtgaagtcgattacttcagaagttccagcagctaaagaggaagttaaaccaactcagacgtcagtcagtcaacaacagtatcaccagcttctgttgccgctgaaacaccagctccagtagctaaagtagcaccggtaagaactgtagcagcccctagagtggcaagtgttaaagtagtcactcctaaagtagaaactggtgcatcaccagagcatgtatcagctccagcagttcctgtgactacgacttcaacagctacagacagtaagttacaagcgactgaagttaagagcgttccggtagcacaaaaagctccaacagcaacaccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcaggtgatccaggtgatcatggtaaaggtttagcagtcgactttattgtaggtaaaaaccaagcacttggtaatgaagttgcacagtactctacacaaaatatggcagcaaataacatttcatatgttatctggcaacaaaagttttactcaaatacaaatagtatttatggacctgctaatacttggaatgcaatgccagatcgtggtggcgttactgccaaccattatgaccatgttcacgtatcatttaacaaataa

SEQ ID NO: 9 - DNA, 80 bp, Streptococcus agalactiae
gtgcatcaccagagcatgtatcagctccagcagttcctgtgactacgacttcaacagctacagacagtaagttacaagcg

SEQ ID NO: 10 - DNA, 29 bp, Artificial Sequence
  probe
agctccagcagttcctgtgactacgactt

(19) United States
(12) Patent Application Publication
(10) Pub. No.: US 2006/0252064 A1
(43) Pub. Date: Nov. 9, 2006

(54) Polynucleotide primers and probes for rapid detection of group B streptococcus (GBS)

(76) Inventors: Betty Wu, Canton, MI (US); Purnima Kurnool, Canton, MI (US)

Correspondence Address: KLAUBER & JACKSON, 411 HACKENSACK AVENUE, HACKENSACK, NJ, 07601, US

(21) Appl. No.: 11/360,855

(22) Filed: Feb. 23, 2006

Related U.S. Application Data
(63) Continuation-in-part of application No. 10/102,513, filed on Mar. 20, 2002.

(51) Int. Cl. C12Q 1/68; C12P 19/34

(52) U.S. Cl. 435/6; 435/91.2

(57) ABSTRACT

The present invention relates to highly specific oligonucleotide primers and probes useful in a rapid and specific method for detecting the presence of Group B Streptococcal (GBS) or Streptococcus agalactiae infection in a biological sample.

### FIELD OF THE INVENTION

This invention relates to methods for detecting Group B streptococcal (GBS) infections, particularly to methods allowing a rapid and accurate diagnosis to prevent and treat neonatal GBS infections.

### BACKGROUND

Group B streptococci (GBS) are responsible for a broad range of severe human diseases, predominantly the life-threatening bacterial infections in neonates and very young infants. Approximately 70 to 80% of infant infections occur in the first few days of life, designated so-called early-onset disease, while late-onset infections occur in infants between 1 week and 3 months of age. Newborns with early-onset GBS disease usually acquire the organism during delivery from their GBS-colonized mothers, manifesting in sepsis and meningitis which cause not only illness and death, but long term disabilities such as hearing loss, impaired vision, developmental problems, and cerebral palsy.

In order to substantially reduce the incidence of early-onset GBS disease, prenatal screening for GBS and intrapartum antimicrobial prophylaxis are now highly recommended in the United States. However, since these strategies require the frequent use of antibiotics, antibiotic resistant GBS or other bacterial agents might emerge during the perinatal period. In addition, these measures are unlikely to prevent late-onset infections, prematurity, and stillbirths related to GBS, while obviously not addressing GBS disease in nonpregnant adults. GBS are increasingly recognized as a frequent cause of invasive infections in pregnant women and clinically ill and older adults, such as those suffering from diabetes, cirrhosis, malignancies and immunodeficiencies.

Currently, culture, including broth culture in selective medium, is the gold standard method for detection of GBS. However, the culture methods require up to 36 hours to obtain results and predict only 87% of women likely to be colonized by GBS at delivery. A rapid, sensitive, and specific test for detection of GBS directly from clinical specimens would allow for a simpler and more efficient prevention program.

Rapid tests have been developed, such as the rapid antigen-based tests, but these tests are neither sensitive nor specific enough to substitute for bacterial culture. The most widely used hybridization-based test to date is the Accuprobe Group B Streptococcocus Identification Test™ (Gen-Probe, San Diego, Calif.). This test is used to detect the presence of GBS in culture media. Although this test only takes about 45 minutes to complete, a pre-incubation period of 18-24 hours in selective broth media is necessary to allow for the growth amplification needed to achieve a satisfactory level of sensitivity. Another frequently used test is the Affirm GBS Microbial Identification System (Micro Probe, Bothel, Wash.). Although this test is reported to be highly specific and can be completed in about 50 minutes, this test demonstrates a level of sensitivity of only about 8.3% in colonized women. The sensitivity increases to about 86% after a 16-24 hour preincubation period. Thus, such a test offers no benefit over culture methods.

Although the above methods can be used to detect GBS, there is an urgent need for a rapid, sensitive, specific, user friendly and reliable method for detecting GBS in patient samples. The present invention provides probes, primers and methods for detecting the specific GBS genes that meet these needs.

Bergeron et al. (WO 98/20157) teach a method of detecting the presence of Streptococcus agalactiae using primers and probes specific for Streptococcal agalactiae, in particular base pairs 58-91 and 190-212 of SEQ ID NO: 30. Bergeron et al. do not teach primers suitable for using in a real time PCR or Taqman procedure such as those of the instant invention. The amplicon is too large to use in a real time PCR or Taqman PCR procedure. Such a real time PCR requires primers having an amplicon no larger than 150 base pairs. Brodeur et al. (WO 99/42588) teach various polynucleotide sequences primarily for preparing vaccine compositions. Brodeur et al. also teach using SEQ ID NO: 42 for detecting group B streptococci in biological samples. Brodeur et al. do not teach that SEQ ID 42 is part of the sip gene. Moreover, Brodeur et al. do not teach primers that are specific to Group B streptococci. Hassan et al., Can. J. Microbiol. Vol. 46, pp. 946-951, teach detecting streptococcus using primers that amplify regions of the cfb gene and a second set of primers based on the V2 region of the 16S rRNA gene of S. agalactiae, and a species specific part of the 16S-23S rRNA intergenic spacer region. Hassan et al. do not teach using the primers of the present application. Furthermore, the primers used by Hassan et al. are not suitable for use as real time PCR primers such as those used according to the above-referenced application. The amplicon is too large to use in a real time PCR procedure (larger than 150 base pairs). Buck et al., Biotechniques, Vol. 27, pp. 528, 1999 teach that any primer stands a reasonable chance of success when primer performance was tested for DNA sequencing. This assumption might be true in the context of purified DNA. However, selecting primers for specifically detecting group B streptococci from clinical isolates is not as simple as Buck et al. propose. Buck et al. do not teach using primers specific for a group B streptococci cAMP or sip gene. Podbielski et al. teach cloning of the cAMP factor using degenerate primers with sequences derived from the CAMP factor amino acid sequence of GBS strain NCTC8181. Podbielski et al., Med. Microbiol. Immunol. Vol,. 183, pp. 239, 1995 do not teach the primers of the present invention. Podbielski et al. do not teach or suggest that cAMP primers might be used alone or in combination with sip primers to detect group B streptococci.

### SUMMARY OF THE INVENTION

In a first aspect, the present invention features a rapid and accurate PCR-based assay for Streptococcus agalactiae, the organism responsible for neonatal Group B Streptococcal (GBS) infections.

The invention includes a pair of hybridization primers (SEQ ID NOs: 1 and 2); or (SEQ ID NOs: 1 and 11) specific to a portion of the cfb gene (FIG. 1; SEQ ID NO: 3) between positions 328 and 451 (SEQ ID NO:4) encoding the CAMP factor (named after Christie, Atkins and Munch-Petersen). The CAMP factor is a diffusible extracellular protein and is produced by the majority of GBS strains.

Further, the instant invention provides a specific probe (SEQ ID NO: 5) designed to recognize the sequence amplified between the primers, e.g., the amplicons of the cfb gene comprised of the 123 bp sequence of SEQ ID NO:4, allowing real-time detection by using fluorescence measurements.

The present invention also includes a pair of GBS specific PCR amplification primers (SEQ ID NO: 6 and 7) specific for a portion of the sip gene (FIG. 2; SEQ ID NO:8) between positions 778 and 857 (SEQ ID NO:9). GBS sip gene (GenBank Accession Numbers: AF151357, AF151358, AF151359, AF151360, AF151361, AF151362) encodes a 53-kDa protein called surface immunogenic protein (“Sip”), which is present in all GBS serotypes. Further included is a specific probe (SEQ ID NO: 10) which recognizes the amplicons allowing real-time detection by using fluorescence measurement.

Accordingly, in one aspect, the invention features a method of determining the presence of Streptococcus agalactiae, comprising:
(a) isolating a sample from a patient;
(b) incubating the sample with a pair of CAMP-based Group B Streptococcal (GBS)-specific primers under conditions wherein the GBS-specific primers hybridize to GBS-related nucleic acids in the sample;
(c) amplifying the GBS-related nucleic acids in the sample by polymerase chain reaction; and
(d) detecting the presence of GBS-related nucleic acids, wherein detection of GBS-related nucleic acids in the sample is a positive indicator of Streptococcus agalactiae infection.
   Optionally, the method may comprise in step (b) incubating the sample with (i) a first pair of CAMP-based Group B Streptococcal (GBS)-specific primers, and (ii) a second pair of Sip-based GBS-specific primers under conditions wherein the GBS-specific primers hybridize to GBS-related nucleic acids in the sample.

In one embodiment, the pair of CAMP-based GBS primers are the oligonucleotides of SEQ ID NOs:1, 2, AND 11. In another embodiment, the second pair of Sip-based GBS primers are the oligonucleotides of SEQ ID NOs:5 and 6. In a further embodiment, the first pair of CAMP-based GBS primers are the oligonucleotides of SEQ ID NOs:1, 2, 11 and the second pair of Sip-based GBS primers are the oligonucleotides of SEQ ID NOs:6 and 7. Mixtures of the above noted primers are envisioned for use in the PCR reaction methods described in the present invention.

In one embodiment, the method of the invention is used in conjunction with SYGR as a means of amplicon detection. SYBR is a fluorescent dye which binds to double stranded DNA and fluoresces strongly when bound to double stranded DNA. In another embodiment, step (a) is conducted in the presence of the probe of SEQ ID NOs:5. In another embodiment, step (a) is conducted in the presence of the probe of SEQ ID NO:10. In a more specific embodiment, step (a) is conducted in the presence of the probe of SEQ ID NOs:5. In another embodiment, step (a) is conducted in the presence of the probes of SEQ ID NOs:5 and 10.

The probes of SEQ ID NOs:5 and 10 are double labeled with a fluorophore at the 5′ and a quencher at the 3′, so when the probe is intact the flourophore is not able to fluorescence (TaqMan®Probe, IDT, Coralville, Iowa). During PCR extension, the probes hybridized to the amplicons is cleaved by the 5′-3′ exonuclease activity of Taq polymerase, resulting in release of the 5′ fluorophore from its quencher. The intensity of the fluorescence increases as more amplicons are synthesized.

In a further embodiment, the sample is a biological sample obtained from a patient to be tested for the presence of GBS. In accordance with the methods of the present invention, such biological samples comprise nucleic acids. DNA may be extracted from any biological sample by any known method in accordance with the method of the present invention. Biological samples include, for example, vaginal or anal specimens, amniotic fluid, spinal fluid, or plasma.

In a further embodiment, step (a) is conducted in a volume of 0.2-100 μl; in further embodiments, the reaction of step (a) is conducted in a volume of less than 50 μl, or less than 25 μl; in a still further embodiment, the reaction is conducted in less than 15 μl.

In a second aspect, the invention features a method of diagnosing a Group B Streptococcal (GBS) infection, comprising:

(a) isolating a sample from a patient

(a) contacting a GBS-related target nucleic acid with a pair of CAMP-based Group B Streptococcal (GBS)-specific primers and a CAMP-based Group B Streptococcal (GBS)-specific probe under conditions wherein GBS-related nucleic acids are amplified; and

(b) detecting the amplified products, wherein detection of amplified products indicates the presence of a GBS infection.

In a third aspect, the invention features an in vitro method for detecting the presence of Streptococcus agalactiae in a biological sample, comprising:

(a) performing PCR in a total volume of between 0.2-100 μl in the presence of a pair of primers comprising SEQ ID NOs:1 and 2, and labeled probes comprising SEQ ID NO:10, under conditions wherein the presence of a Streptococcus agalactiae-related nucleic acid sequence results in an amplified and labeled PCR product; and

(b) detecting the presence of PCR product with either specific probes or SYBR, wherein detection of a Streptococcus agalactiae specific PCR product is a positive indicator for Streptococcus agalactiae infection.

In a fourth aspect, the invention features a method for detecting a Group B Streptococcal (GBS) infection in a patient, comprising:

(a) obtaining a biological sample from the patient;

(b) performing PCR in the presence of a first pair of primers comprising SEQ ID NOs:1 and 2, or SEQ ID NOs: 1 and 11 and labeled probes comprising SEQ ID NO: 5 under conditions wherein the presence of a Streptococcus agalactiae-related nucleic acid sequence results in an amplified and labeled PCR product; and

(c) detecting the presence of PCR product with specific fluorescent labeled probes or SYBR, wherein the presence of degraded probe indicates the presence of a GBS infection.

In a fifth aspect of the invention, kits are provided for detecting the presence of GBS genes in a biological sample, as described in the invention herein. The kits contain one or more components used in the methods of this invention, and may contain instructions for use. In addition to the specific components listed below, the kits may contain other components, such as a disposable microfluidic PCR chip useful to perform the methods of the invention, such as Taq DNA polymerase, or reverse transcriptase in the case for rt-PCR, PCR buffers, four deoxyribonucleotides triphosphates (adenosine, cytosine, guanine, and thymine); and, or uracil ribonucleotide triphosphates, and other components known to the art. Thus, the invention includes a kit for amplifying all or a portion of at least one target nucleic acid in a sample containing a plurality of DNAs, each kit comprising one or more containers: The primers and probes may be in a separate compartment than that of other PCR reagents. The reagents and primers and probes may be in either liquid form, or frozen, or in a lyophilized form.

A specific embodiment includes a kit wherein the probes are specific for the gene encoding CAMP of GBS. Optionally, the kit may contain probes specific for the genes encoding CAMP and Sip of GBS.

These and other aspects of the present invention will become evident upon reference to the following detailed description and attached drawings. In addition, various references are set forth herein which describe in more detail certain procedures or compositions and are therefore incorporated by reference in their entirety.

### BRIEF DESCRIPTION OF THE DRAWINGS

FIG. 1 is the cfb gene (SEQ ID NO: 3) showing primers (SEQ ID NOs: 1 and 2) specific to the portion of the cfb gene between positions 328 and 451 (SEQ ID NO:4) encoding the CAMP factor, and a specific probe (SEQ ID NO: 5) designed to recognize the sequence amplified between the primers comprised of the 123 bp sequence of SEQ ID NO:4.

FIG. 2 is the sip gene (SEQ ID NO:8) with the positions of the primers (SEQ ID NO: 6 and 7) specific for a portion of the sip gene between positions 778 and 857 (SEQ ID NO:9) and a specific probe (SEQ ID NO: 10) recognizing the amplicons.

### DETAILED DESCRIPTION

Before the present methods and compositions are described, it is to be understood that this invention is not limited to particular methods, compositions, and experimental conditions described, as such methods and compounds may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

Definitions

An “Amplicon” is a nucleic acid sequence amplified by the specific primers during the course of a polymerase chain reaction (PCR), i.e., the fragment produced by PCR amplification using a primer pair of the present invention. In the instances where the amplicon is the product of real time PCR, the amplicon is preferably about 150 base pairs or less in length.

The “CAMP” (Christie-Atkins-Munch-Petersen) factor is a diffusible extracellular protein and is produced by the majority of GBS. The gene encoding CAMP factor, the cfb gene (SEQ ID NO: 3) (GenBank access number: X72754), is present in virtually every GBS isolate.

The GBS “sip” gene (SEQ ID NO:8) (GenBank access number: AF151357, AF151358, AF151359, AF151360, AF151361, AF151362) encodes a 53-kDa protein called surface immunogenic protein (“Sip”), which is present in all serotypes of GBS.

As used herein, “label” or “labeled moiety capable of providing a signal” refers to any atom or molecule which can be used to provide a detectable (preferably quantifiable) signal, and which can be operatively linked to a nucleotide or nucleic acid. Labels may provide signals detectable by fluorescence, radioactivity, colorimetry, gravimetry, X-ray diffraction or absorption, magnetism, enzymatic activity, mass spectrometry, binding affinity, hybridization radiofrequency and the like.

As used herein, “sample” refers to any substance containing or presumed to contain a nucleic acid of interest (a target nucleic acid sequence such as the genes of the present invention found in Group B Streptococcus, including the cfb and sip genes) or which is itself a nucleic acid containing or presumed to contain a target nucleic acid sequence of interest. The term “sample” thus includes a sample of nucleic acid (genomic DNA, cDNA, RNA), cell, organism, tissue, fluid, or substance including but not limited to, for example, vaginal or anal swabs, amniotic fluid, whole blood, plasma, serum, spinal fluid, urine, stool, intestinal and genitourinary tracts, blood cells, samples of in vitro cell culture constituents, microbial specimens, and objects or specimens that have been “marked” with nucleic acid tracer molecules.

As used herein, “target nucleic acid sequence” refers to a region of a nucleic acid that is to be either replicated, amplified, and/or detected. In one embodiment, the “target nucleic acid sequence” resides between two primer sequences used for amplification. In other cases the target may be a nucleic acid that is not amplified.

As used herein, “nucleic acid polymerase” refers to an enzyme that catalyzes the polymerization of nucleoside triphosphates. Generally, the enzyme will initiate synthesis at the 3′-end of the primer annealed to the target sequence, and will proceed in the 5′-direction along the template, and if possessing a 5′ to 3′ nuclease activity, it may also hydrolyze intervening, annealed probe to release both labeled and unlabeled probe fragments, until synthesis terminates. Known DNA polymerases include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase.

As used herein, “5′ to 3′exonuclease activity” refers to that activity of a template-specific nucleic acid polymerase, e.g. a 5′ to 3′ exonuclease activity traditionally associated with some DNA polymerases whereby mononucleotides or oligonucleotides are removed from the 5′ end of a polynucleotide in a sequential manner, (i.e., E. coli DNA polymerase I has this activity whereas the Klenow (Klenow et al., 1970, Proc. Natl. Acad. Sci., USA, 65:168) fragment does not, (Klenow et al., 1971, Eur. J. Biochem., 22:371)), or polynucleotides are removed from the 5′ end by an endonucleolytic activity that may be inherently present in a 5′ to 3′ exonuclease activity.

As used herein, “endonuclease” refers to an enzyme that cleaves bonds, preferably phosphodiester bonds, within a nucleic acid molecule. An endonuclease according to the invention can be specific for single-stranded or double-stranded DNA or RNA.

As used herein, “exonuclease” refers to an enzyme that cleaves bonds, preferably phosphodiester bonds, between nucleotides one at a time from the end of a polynucleotide. An exonuclease according to the invention can be specific for the 5′ or 3′ end of a DNA or RNA molecule, and is referred to herein as a 5′ exonuclease or a 3′ exonuclease.

As used herein, “detecting a target nucleic acid sequence” refers to determining the presence of a particular target nucleic acid sequence in a sample or determining the amount of a particular target nucleic acid sequence in a sample as an indication of the presence of a target nucleic acid sequence in a sample. The amount of a target nucleic acid sequence that can be measured or detected is preferably about 1 molecule to 10²⁰ molecules, more preferably about 100 molecules to 10¹⁷ molecules and most preferably about 1000 molecules to 10¹⁴ molecules. Preferably there is a direct correlation between the amount of the target nucleic acid sequence and the signal generated by the detected nucleic acid.

As used herein, an “oligonucleotide primer” refers to a single stranded DNA or RNA molecule that is hybridizable (eg. capable of annealing) to a nucleic acid template and is capable of priming enzymatic synthesis of a second nucleic acid strand. Alternatively, or in addition, oligonucleotide primers, when labeled directly or indirectly (e.g., bound by a labeled secondary probe which is specific for the oligonucleotide primer) may be used effectively as probes to detect the presence of a specific nucleic acid in a sample. Oligonucleotide primers useful according to the invention are between about 10 to 100 nucleotides in length, preferably about 17-50 nucleotides in length and more preferably about 17-40 nucleotides in length and more preferably about 17-30 nucleotides in length. Oligonucleotide probes useful for the formation of a cleavage structure according to the invention are between about 17-40 nucleotides in length, preferably about 17-30 nucleotides in length and more preferably about 17-25 nucleotides in length.

As used herein, “template dependent polymerizing agent” refers to an enzyme capable of extending an oligonucleotide primer in the presence of adequate amounts of the four deoxyribonucleoside triphosphates (dATP, dGTP, dCTP and dTTP) or analogs as described herein, in a reaction medium comprising appropriate salts, metal cations, appropriate stabilizers and a pH buffering system. Template dependent polymerizing agents are enzymes known to catalyze primer- and template-dependent DNA synthesis, and possess 5′ to 3′ nuclease activity. Some of which possess 5′ to 3′ nuclease activity.

As used herein, “amplifying” refers to the generation of additional copies of a nucleic acid sequence. A variety of methods have been developed to amplify nucleic acid sequences, including the polymerase chain reaction (PCR). PCR amplification of a nucleic acid sequence generally results in the exponential amplification of a nucleic acid sequence(s) and or fragments thereof.

By “homologous” is meant a same sense nucleic acid which possesses a level of similarity with the target nucleic acid within reason and within standards known and accepted in the art. With regard to PCR, the term “homologous” may be used to refer to an amplicon that exhibits a high level of nucleic acid similarity to another nucleic acid, e.g., the template cDNA. As is understood in the art, enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplified nucleic acid (i.e., amplicon) need not be completely identical in every nucleotide to the template nucleic acid.

“Complementary” is understood in its recognized meaning as identifying a nucleotide in one sequence that hybridizes (anneals) to a nucleotide in another sequence according to the rule A→T, U and C→G (and vice versa) and thus “matches” its partner for purposes of this definition. Enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplicon need not be completely matched in every nucleotide to the target or template RNA.

As used herein, the terms “nucleic acid”, “polynucleotide” and “oligonucleotide” refer to primers, probes, and oligomer fragments to be detected, and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including abasic sites). There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single-stranded RNA.

The term “primer” may refer to more than one primer and generally refers to an oligonucleotide, whether occurring naturally, as in a purified restriction digest, or produced synthetically, which is capable of acting as a point of initiation of DNA synthesis when annealed to a nucleic acid template and placed under conditions in which synthesis of a primer extension product which is complementary to the template is catalyzed. Such conditions include the presence of four different deoxyribonucleoside triphosphates and a polymerization-inducing agent such as a DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification.

The “polymerase chain reaction (PCR)” technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment (i.e, an amplicon) whose termini are defined by the 5′ ends of the primers. PCR is reported to be capable of producing a selective enrichment of a specific DNA sequence by a factor of 10⁹. The PCR method is also described in Saiki et al., 1985, Science, 230:1350.

As used herein, “probe” refers to a labeled oligonucleotide primer, which forms a duplex structure with a sequence in the target nucleic acid, due to complementarity of at least one sequence in the probe with a sequence in the target region. Such probes are useful for identification of a target nucleic acid sequence for GBS according to the invention, including the CAMP and Sip genes of GBS. Pairs of single-stranded DNA primers can be annealed to sequences within a target nucleic acid sequence or can be used to prime DNA synthesis of a target nucleic acid sequence.

The method described herein is a rapid and accurate screening test for the presence of GBS in a biological sample. In a particular aspect of the invention, a biological sample may be a bodily fluid derived from a pregnant female. Such a biological sample may be isolated prior to or at the time of delivery. Isolation of a biological sample at the time of birth obviates the need for prenatal screening which may involve potential risk to the morbidity and/or mortality of the mother and/or fetus. Moreover, in that the methods of the present invention provide a rapid and definitive positive indicator for the presence of GBS infection, they will also dramatically reduce the inappropriate use of antibiotic prophylaxis in women who are not colonized.

Standard molecular biology techniques known in the art and not specifically described herein may be found in a variety of standard laboratory manuals including: Sambrook et al., Molecular Cloning: A Laboratory Manual, Cold Springs Harbor Laboratory, New York (1992).

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

The present invention features a rapid and accurate PCR-based assay for Streptococcus agalactiae, the organism responsible for neonatal Group B Streptococcal (GBS) infections. Furthermore, the present invention identifies and utilizes specific primers and probes specific for the cfb and, optionally, the sip genes in GBS, which can be utilized in various PCR assays for specific and rapid identification of GBS in biological samples. The specific primers so identified can be used as a mixture to aid in increasing the sensitivity of screening for GBS using PCR. Moreover, the primers and probes identified herein can be used in real time PCR and in on-chip PCR assays for rapid and convenient identification of GBS in clinical samples. The use of these primers and probes in on-chip PCR reactions allows for portable testing and eliminates the need for testing in a central laboratory.

The gene encoding CAMP factor, cfb gene (GenBank access number: X72754), is present in virtually every GBS isolate, a feature which has been used to advantage for the development of a PCR based assay for GBS contamination as described herein. (Danbing K. et al., 2000, Clinical Chemistry, 46, 324-331). The present invention, however, is directed to the use of novel methods and specific GBS primers which represents a substantial improvement as compared to the methods disclosed by Danbing et al. The present invention encompasses the use of specific primers or primer mixtures to aid in an increase in sensitivity and specificity of the screening or diagnostic test method.

GBS-specific polymerase chain reaction (PCR) assays have been described which provide improved detection of GBS (Bergeron et al. (2000) N. Engl. J. Med. 343, 175-179). The technique used by Bergeron et al. requires rather sophisticated machinery and the transport of the specimen to a lab, which would require constant staffing with significant training to run the specimen. These factors may result in unacceptable delays in implementing effective and timely antibiotic therapy. Moreover, the implementation of this particular technology in a labor and delivery unit would be dependent on a fairly large service to justify the financial cost of personnel providing this service. Because the service would have to be available 24 hours a day, most obstetrical services in this country would likely not be able to employ it. The aspects of the present invention provide an advantage over that described by Bergeron in that the approach used herein is more robust when used in real-time PCR reactions. Furthermore, the combined use of primer mixtures with on-chip PCR allows for increased sensitivity and specificity of the reaction, as well as providing the ability to diagnose the infection as GBS immediately (on site) due to the use of the on-chip PCR assay, which allows for portable use.

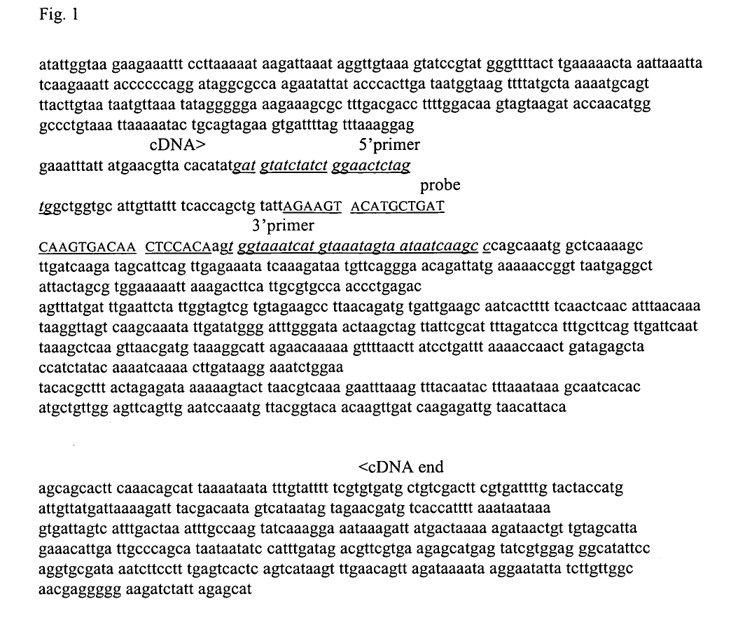

In preferred embodiments, the methods of the present invention use a pair of hybridization primers (SEQ ID NO: 1, 2, and 11) specific to the portion of the cfb gene (FIG. 1; SEQ ID NO: 3) between positions 328 and 451 (SEQ ID NO:4) encoding the CAMP factor (named after Christie, Atkins and Munch-Petersen). The CAMP factor is a diffusible extracellular protein and is produced by the majority of GBS. The gene encoding CAMP factor, cfb gene (GenBank access number: X72754), is present in virtually every GBS isolate and has been used for the development of a PCR based identification of GBS (Danbing K. et al., 2000, Clinical Chemistry, 46, 324-331).

Further, in preferred embodiments, the instant invention also utilizes a specific probe (SEQ ID NO: 5) designed to recognize the sequence amplified between the primers, e.g., the amplicons of the cfb gene comprised of the 123 bp sequence of SEQ ID NO:4, allowing real-time detection by using fluorescence measurements. In preferred embodiments, the amplicon is less than or equal to about 150 base pairs in length.

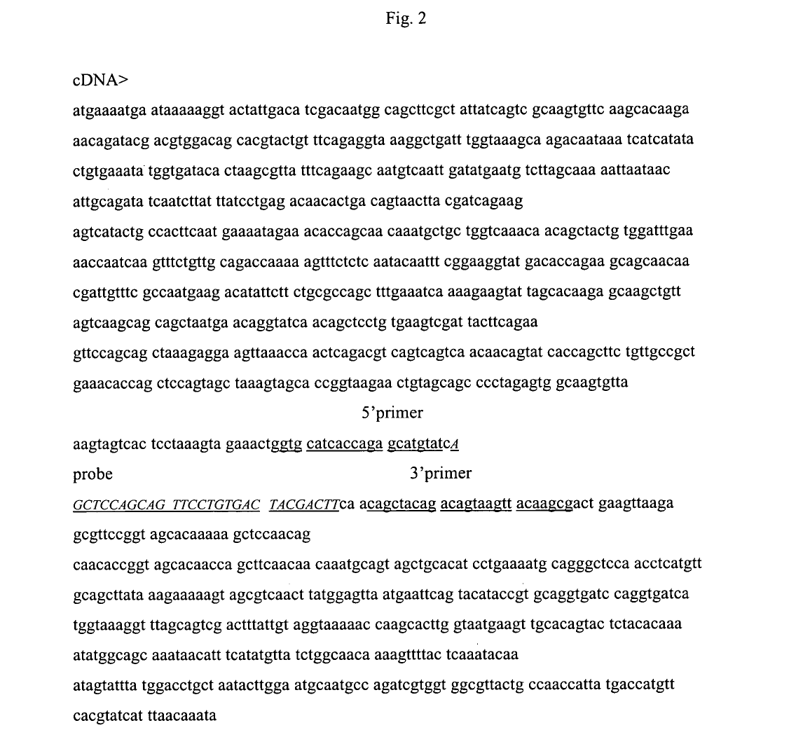

Optionally, the present invention may use a pair of GBS specific PCR amplification primers (SEQ ID NO: 6 and 7) specific for a portion of the sip gene (FIG. 2; SEQ ID NO:8) between positions 778 and 857 (SEQ ID NO:9). GBS sip gene (GenBank access number: AF151357, AF151358, AF151359, AF151360, AF151361, AF151362) encodes a 53-kDa protein called surface immunogenic protein (“Sip”), which is present in all serotypes of GBS. Further included is a specific probe (SEQ ID NO: 10) recognizing the amplicons and allowing real-time detection by using fluorescence measurement. In preferred embodiments, the amplicon is less than or equal to about 150 base pairs in length.

Accordingly, in a first aspect, the invention features a method for detecting Streptococcus agalactiae, comprising:
(a) hybridizing a sample obtained from a patient suspected of being infected with Group B Streptococcal (GBS), with a pair of CAMP-based Group B Streptococcal (GBS)-specific primers, and, optionally, with a second pair of Sip-based GBS-specific primers;
(b) amplifying the GBS-related nucleic acids; and
(c) detecting the presence of GBS-related nucleic acids.

In one embodiment, the pair of CAMP-based GBS primers are the oligonucleotides of SEQ ID NOs:1 and 2; or of oligonucleotides of SEQ ID NOs: 1 and 11. In another embodiment, the pair of Sip-based GBS primers are the oligonucleotides of SEQ ID NOs: 5 and 6. In a further embodiment, the pair of CAMP-based GBS primers are the oligonucleotides of SEQ ID NOs:1 and 2, or of oligonucleotides of SEQ ID NOs:1 and 11 and the pair of Sip-based GBS primers are the oligonucleotides of SEQ ID NOs:6 and 7.

In one embodiment, the invention is used in conjunction with SYGR as a means of amplicon detection. SYGR is a fluorescent dye which binds to double stranded DNA and fluoresces strongly when bound to double stranded DNA.

In another embodiment, step (a) is conducted in the presence of the probe of SEQ ID NO:5. In another embodiment, step (a) is conducted in the presence of the probe of SEQ ID NO:10. In another embodiment, step (a) is conducted in the presence of the probes of SEQ ID NOs: 5 and 10. The probes of SEQ ID NOs: 5 and 10 may be double labeled with a fluorophore at the 5′ and a quencher at the 3′, so when the probe is intact the flourophore does not emit a detectable fluorescent signal (TaqMan®Probe, IDT, Coralville, Iowa). During PCR extension, the probes hybridized to the amplicons are cleaved by the 5′-3′ exonuclease activity of Taq polymerase, resulting in release of the 5′ fluorophore from its quencher. The intensity of the fluorescence increases as a function of the synthesis of additional amplicons during the course subsequent cycles of PCR.

The biological sample may be obtained from a patient to be tested for the presence of GBS. Such biological samples may include nucleic acid sequences detectable using the methods of the invention. DNA from any biological sample extracted by any known method may be used in the method of the invention. Biological samples include, for example, vaginal or anal specimens, amniotic fluid, spinal fluid, whole blood, serum or plasma.

In some embodiments, step (a) is conducted in a volume of 0.2-100 μl. In further embodiments, the reaction of step (a) is conducted in a volume of less than 50 μl, or less than 25 μl. In still further embodiments, the reaction is conducted in less than 15 μl.

In a second aspect, the invention features a method of diagnosing a Group B Streptococcal (GBS) infection, comprising:

(a) contacting a GBS-related target nucleic acid with a pair of CAMP-based Group B Streptococcal (GBS)-specific primers, and a CAMP-based Group B Streptococcal (GBS)-specific probe, and optionally, a pair of Sip-based GBS-specific primers, and a Sip-based GBS-specific probe, under conditions wherein GBS-related nucleic acids are amplified; and

(b) detecting the amplified products, wherein detection of amplified products indicates the presence of a GBS infection.

In a third aspect, the invention features an in vitro method for detecting the presence of Streptococcus agalactiae in a biological sample, comprising:

(a) releasing nucleic acids from said biological sample;

(b) performing PCR in a total volume of between 0.2-100 μl in the presence of a first pair of primers comprising SEQ ID NOs:1 and 2, or SEQ ID Nos: 1 and 11, or combinations thereof and, optionally, a second pair of primers comprising SEQ ID NOs:6 and 7, and one or more labeled probes comprising SEQ ID NO:5 and, optionally SEQ ID NO:10, under conditions wherein the presence of a Streptococcus agalactiae-related nucleic acid sequence results in an amplified and labeled PCR product; and

(c) detecting the presence of PCR product with either specific probes or SYBR.

In a fourth aspect, the invention features a method for detecting a Group B Streptococcal (GBS) infection in a patient, comprising:

(a) obtaining a biological sample from the patient;

(b) releasing nucleic acids from said biological sample;

(c) performing PCR in a total volume of between 0.2-100 μl in the presence of a pair of primers comprising SEQ ID NOs:1 and 2, or SEQ ID NOs: 1 and 11, or a combination thereof, and, optionally, a pair of primers comprising SEQ ID NOs:6 and 7, and a labeled probe comprising SEQ ID NO:5 and, optionally, SEQ ID NO: 10, under conditions wherein the presence of a Streptococcus agalactiae-related nucleic acid sequence results in an amplified and labeled PCR product; and

(d) detecting the presence of the PCR product with a specific fluorescent labeled probe or SYBR, wherein the presence of a degraded probe indicates the presence of a GBS infection.

In a fifth aspect of the invention, kits are provided for detecting the presence of GBS isolates or genes in a biological sample, as described herein. The kits contain one or more components used in the methods of this invention, and may contain instructions for use. In addition to the specific components listed below, the kits may contain other components useful for performing the methods of the invention, such as RNA or DNA polymerase, buffers, reagents, and other components known to the art. Thus, the invention includes a kit for amplifying all or a portion of at least one target nucleic acid in a sample containing a plurality of DNAs, each kit comprising one or more containers: (a) a primer for first-strand cDNA synthesis comprising a sequence which anneals to a selected nucleotide sequence of the target nucleic acid sequence (e.g., mRNA); (b) a primer for second-strand cDNA synthesis which produces a second-strand cDNA comprising either an RNA polymerase promoter at the 5′ end of its sense strand, or a PCR primer site at the 5′ end of its antisense strand, or both (for the same sense method) or a PCR primer site at the 5′ end of its sense strand, or an RNA polymerase promoter at the 5′ end of its antisense strand or both (for the antisense method); (c) a first PCR primer comprising an RNA polymerase promoter sequence; and (d) a second PCR primer comprising a PCR primer site sequence; (e) adenosine, cytosine, guanine, and thymine deoxyribonucleotide triphosphates; and (f) adenosine, cytosine, guanine, and uracil ribonucleotide triphosphates.

A specific embodiment of a kit of the invention may also include one or more probes specific for a gene encoding CAMP or Sip of GBS. As described herein, such probes may be labeled (e.g., fluorescently labeled) to facilitate their use real time detection of amplicons produced during the course of PCR amplification.

Thus, as noted above, methods are provided for determining the presence of a GBS gene in a biological sample. The particular genes of interest in the present invention are the cfb gene (SEQ ID NO: 3) and sip gene (SEQ ID NO: 8). Utilizing such methods as described herein, one of skill in the art can generate accurate and rapid results, which can provide same day results from test samples. This allows more appropriate utilization of antibiotics for the treatment of patients in need thereof. Furthermore, such methods may be readily utilized to monitor outbreaks or for routine surveillance in both nosocomial and non-nosocomial settings.

Such methods may be utilized to detect the presence of a desired target nucleic acid molecule (e.g. for GBS) within a biological sample. Representative examples of biological samples include cultured (e.g., samples grown in a bacteriological medium) or clinical samples, including for example, samples from vaginal or anal swabs, whole blood, serum, plasma, urine, stool, and abscess or spinal fluids. Methods for generating target nucleic acid molecules may be readily accomplished by one of ordinary skill in the art given the disclosure provided herein and general knowledge of such procedures (see generally, Sambrook et al., Molecular Cloning: A Laboratory Manual (2d ed.), Cold Spring Harbor Laboratory Press, 1989).

As noted above, within one aspect of the present invention the target nucleic acid molecule is reacted with a complementary single-stranded nucleic acid probe. Preferably, probes are designed which hybridize with the cfb gene encoding CAMP and, optionally, the sip gene encoding the surface immunogenic protein of GBS.

Although within various embodiments of the invention a single-stranded probe is utilized to react or hybridize to a single-stranded target sequence, the above-described methods should not be limited to situations wherein complementary probe and target sequences pair to form a duplex.

Single stranded nucleic acid molecules may be synthesized or obtained and/or prepared directly from a target cell or organism utilizing standard techniques (see, e.g., Sambrook et al., “Molecular Cloning: A Laboratory Manual”, Cold Spring Harbor, 1989), or prepared utilizing any of a wide variety of a techniques, including for example, PCR, NASBA reverse transcription of RNA, SDA branched-chain DNA and the like.

The DNA or RNA molecules utilized may be derived from naturally occurring sources, or they may be synthetically formed. Each may be from about 5 bases to 10,000 bases in length. Within certain variants, the probe and target nucleic acid molecule need not be perfectly complementary, and indeed, may be purposely different by one, two, three or more nucleic acids (see, e.g., PCT Publication WO 95/14106 and U.S. Pat. No. 5,660,988). Within further variants, the target nucleic acid molecule is present in a heterogeneous population of genomic nucleic acids.

Nucleic Acid Sequences Useful in the Invention

The invention provides for methods of detecting or measuring a target nucleic acid sequence; and also utilizes specific oligonucleotide primers for amplifying a particular template nucleic acid sequence and specific probes for identifying the target sequence. The complement of a nucleic acid sequence as used herein refers to an oligonucleotide which, when aligned with the nucleic acid sequence such that the 5′ end of one sequence is paired with the 3′ end of the other, is in “antiparallel association.” Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength, the temperature, and incidence of mismatched base pairs.

The oligonucleotide is not necessarily physically derived from any existing or natural sequence but may be generated in any manner, including chemical synthesis, DNA replication, reverse transcription or a combination thereof. Because mononucleotides are reacted to make oligonucleotides in a manner such that the 5′ phosphate of one mononucleotide pentose ring is attached to the 3′ oxygen of its neighbor in one direction via a phosphodiester linkage, one end of an oligonucleotide is referred to as the “5′end” if its 5′ phosphate is not linked to the 3′ oxygen of a mononucleotide pentose ring and as the “3′end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends.

When two different, non-overlapping oligonucleotides anneal to different regions of the same linear complementary nucleic acid sequence, and the 3′ end of one oligonucleotide points toward the 5′ end of the other, the former may be called the “upstream” annealed oligonucleotide and the latter the “downstream” annealed oligonucleotide.

Nucleic Acid Probes and Primers

Primers and Probes Useful for Practicing the Methods of the Invention The invention provides specific oligonucleotide primers and probes useful for detecting or measuring a nucleic acid, and for amplifying a template nucleic acid sequence. Oligonucleotide primers useful according to the invention may be single-stranded DNA or RNA molecules that are hybridizable to a template nucleic acid sequence and prime enzymatic synthesis of a second nucleic acid strand. The primer is complementary to a portion of a target molecule present in a pool of nucleic acid molecules. It is contemplated that oligonucleotide primers according to the invention may be prepared by synthetic methods, either chemical or enzymatic. Alternatively, such a molecule or a fragment thereof may be naturally-occurring, and is isolated from its natural source or purchased from a commercial supplier. Oligonucleotide primers and probes are generally 5 to 100 nucleotides in length, ideally from 17 to 40 nucleotides, although primers and probes of different lengths may also be used. Primers for amplification are preferably about 17-25 nucleotides. Primers useful according to the invention are also designed to have a particular melting temperature (Tm) by the method of melting temperature estimation. Commercial programs, including Oligo.™., Primer Design and programs available on the internet, including Primer3 and Oligo Calculator can be used to calculate a Tm of a nucleic acid sequence useful according to the invention. Preferably, the Tm of an amplification primer useful according to the invention, as calculated for example by Oligo Calculator, is preferably between about 45 and 65° C. and more preferably between about 50 and 60° C. Preferably, the Tm of a probe useful according to the invention is 7° C. higher than the Tm of the corresponding amplification primers.

Typically, selective hybridization occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, a region of mismatch may encompass loops, which are defined as regions in which there exists a mismatch in an uninterrupted series of four or more nucleotides.

Numerous factors influence the efficiency and selectivity of hybridization of the primer to a second nucleic acid molecule. These factors, which include primer length, nucleotide sequence and/or composition, hybridization temperature, buffer composition and potential for steric hindrance in the region to which the primer is required to hybridize, will be considered when designing oligonucleotide primers according to the invention.

A positive correlation exists between primer length and both the efficiency and accuracy with which a primer will anneal to a target sequence. In particular, longer sequences have a higher melting temperature (T_{M}) than do shorter ones, and are less likely to be repeated within a given target sequence, thereby minimizing promiscuous hybridization. Primer sequences with a high G-C content or that comprising palindromic sequences tend to self-hybridize, as do their intended target sites, since unimolecular, rather than bimolecular, hybridization kinetics are generally favored in solution. However, it is also important to design a primer that contains sufficient numbers of G-C nucleotide pairings since each G-C pair is bound by three hydrogen bonds, rather than the two that are found when A and T bases pair to bind the target sequence, and therefore forms a tighter, stronger bond. Hybridization temperature varies inversely with primer annealing efficiency, as does the concentration of organic solvents, e.g. formamide, that might be included in a priming reaction or hybridization mixture, while increases in salt concentration facilitate binding. Under stringent annealing conditions, longer hybridization probes, or synthesis primers, hybridize more efficiently than do shorter ones, which are sufficient under more permissive conditions. Stringent hybridization conditions typically include salt concentrations of less than about 1 M, more usually less than about 500 mM and preferably less than about 200 mM. Hybridization temperatures range from as low as 0° C. to greater than 22° C., greater than about 30° C., and (most often) in excess of about 37° C. Longer fragments may require higher hybridization temperatures for specific hybridization. As several factors affect the stringency of hybridization, the combination of parameters is more important than the absolute measure of a single factor.

Oligonucleotide primers can be designed with these considerations in mind and synthesized according to the following methods.

Oligonucleotide Primer Design Strategy

The design of a particular oligonucleotide primer for the purpose of sequencing, PCR, or for use in identifying target nucleic acid molecules of GBS involves selecting a sequence that is capable of recognizing the target sequence, but has a minimal predicted secondary structure. The oligonucleotide sequence binds only to a single site in the target nucleic acid sequence. Furthermore, the Tm of the oligonucleotide is optimized by analysis of the length and GC content of the oligonucleotide. Furthermore, when designing a PCR primer useful for the amplification of genomic DNA, the selected primer sequence does not demonstrate significant matches to sequences in the GenBank database (or other available databases).

The design of a primer is facilitated by the use of readily available computer programs, developed to assist in the evaluation of the several parameters described above and the optimization of primer sequences. Examples of such programs are “Primer Express” (Applied Biosystems), “PrimerSelect” of the DNAStar™. “PrimerSelect” of the DNAStar.™. software package (DNAStar, Inc.; Madison, Wis.), OLIGO 4.0 (National Biosciences, Inc.), PRIMER, Oligonucleotide Selection Program, PGEN and Amplify (described in Ausubel et al., 1995, Short Protocols in Molecular Biology, 3rd Edition, John Wiley & Sons). In one embodiment, primers are designed with sequences that serve as targets for other primers to produce a PCR product that has known sequences on the ends which serve as targets for further amplification (e.g. to sequence the PCR product). If many different target nucleic acid sequences are amplified with specific primers that share a common ‘tail’ sequence’, the PCR products from these distinct genes can subsequently be sequenced with a single set of primers. Alternatively, in order to facilitate subsequent cloning of amplified sequences, primers are designed with restriction enzyme site sequences appended to their 5′ ends. Thus, all nucleotides of the primers are derived from a target nucleic acid sequence or sequences adjacent to a target nucleic acid sequence, except for the few nucleotides necessary to form a restriction enzyme site. Such enzymes and sites are well known in the art. If the genomic sequence of a target nucleic acid sequence and the sequence of the open reading frame of a target nucleic acid sequence are known, design of particular primers is well within the skill of the art. Also encompassed by the present invention are primers which include various tagging moieties, the incorporation of which into an amplicon enables its detection and/or isolation. Such tags are known to practitioners skilled in the art of molecular biology.

It is well known by those with skill in the art that oligonucleotides can be synthesized with certain chemical and/or capture moieties, such that they can be coupled to solid supports. Suitable capture moieties include, but are not limited to, biotin, a hapten, a protein, a nucleotide sequence, or a chemically reactive moiety. Such oligonucleotides may either be used first in solution, and then captured onto a solid support, or first attached to a solid support and then used in a detection reaction. An example of the latter would be to couple a downstream probe molecule to a solid support, such that the 5′ end of the downstream probe molecule comprised a fluorescent quencher. The target nucleic acid could hybridize with the solid-phase downstream probe oligonucleotide, and a liquid phase upstream primer could also hybridize with the target molecule. This would cause the solid support-bound fluorophore to be detectable. Different downstream probe molecules could be bound to different locations on an array. The location on the array would identify the probe molecule, and indicate the presence of the template to which the probe molecule can hybridize.

Synthesis

The primers themselves are synthesized using techniques that are also well known in the art. Methods for preparing oligonucleotides of specific sequence are known in the art, and include, for example, cloning and restriction digest analysis of appropriate sequences and direct chemical synthesis. Once designed, oligonucleotides are prepared by a suitable chemical synthesis method, including, for example, the phosphotriester method described by Narang et al., 1979, Methods in Enzymology, 68:90, the phosphodiester method disclosed by Brown et al., 1979, Methods in Enzymology, 68:109, the diethylphosphoramidate method disclosed in Beaucage et al., 1981, Tetrahedron Letters, 22:1859, and the solid support method disclosed in U.S. Pat. No. 4,458,066, or by other chemical methods using either a commercial automated oligonucleotide synthesizer (which is commercially available) or VLSIPS™ technology.

Probes

The invention provides for probes useful for identifying sequences specific for the CAMP or Sip genes of GBS.

As used herein, the term “probe” refers to a labeled oligonucleotide which forms a duplex structure with a sequence in the target nucleic acid, due to complementarity of at least one sequence in the probe with a sequence in the target region. The probe, preferably, does not contain a sequence complementary to sequence(s) used in the primer extension (s). Generally the 3′ terminus of the probe will be “blocked” to prohibit incorporation of the probe into a primer extension product. “Blocking” can be achieved by using non-complementary bases or by adding a chemical moiety such as biotin or a phosphate group to the 3′ hydroxl of the last nucleotide, which may, depending upon the selected moiety, serve a dual purpose by also acting as a label for subsequent detection or capture of the nucleic acid attached to the label. Blocking can also be achieved by removing the 3′-OH or by using a nucleotide that lacks a 3′-OH such as dideoxynucleotide.

In certain embodiments of the present invention, the polynucleotide sequences provided herein can be advantageously used as probes or primers for nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence disclosed herein will be of particular utility. Longer contiguous identical or complementary sequences, e.g., those of about 20, 30, 40, 50, 100, 200, 500, 1000 (including all intermediate lengths) and even up to full length sequences also be of use in certain embodiments.

The ability of such nucleic acid probes to specifically hybridize to a sequence of interest will enable them to be of use in detecting the presence of complementary sequences in a given sample.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in PCR assays. This would allow a gene product, or fragment thereof, to be analyzed, in various samples, including but not limited to biological samples. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequence set forth in SEQ ID NOs: 1, 2, 3, 4, 5, 6, 7, 8, 9, 10, or 11 or to any continuous portion of the sequence, from about 15-25 nucleotides in length up to and including the full length sequence, that one wishes to utilize as a probe or primer.

Small polynucleotide segments or fragments may be readily prepared by, for example, directly synthesizing the fragment by chemical means, as is commonly practiced using an automated oligonucleotide synthesizer.

For hybridization techniques, a partial sequence may be labeled (e.g., by nick-translation or end-labeling with ³²P) using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186, 1988), which uses restriction enzymes to generate a fragment in a known region of a gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Probes of the present invention may also have one or more detectable markers attached to one or both ends. The marker may be virtually any molecule or reagent which is capable of being detected, representative examples of which include radioisotopes or radiolabeled molecules, fluorescent molecules, fluorescent antibodies, enzymes, or chemiluminescent catalysts. Within certain embodiments of the invention, the probe may contain one or more labels such as a fluorescent or enzymatic label (e.g., quenched fluorescent pairs, or, a fluorescent label and an enzyme label), or a label and a binding molecule such as biotin (e.g., the probe, either in its cleaved or uncleaved state, may be covalently or non-covalently bound to both a label and a binding molecule (see also, e.g., U.S. Pat. No. 5,731,146).

As noted above, the probes of the present invention may also be linked to a solid support either directly, or through a chemical linker. Representative examples of solid supports include silicaceous, cellulosic, polymer-based, or plastic materials.

Methods for constructing such nucleic acid probes may be readily accomplished by one of ordinary skill in the art, given the disclosure provided herein. Particularly preferred methods are described for example by: Matteucci and Caruthers, J. Am. Chem. Soc. 103:3185,1981; Beaucage and Caruthers, Tetrahedron Lett. 22:1859-1862, 1981; U.S. Pat. Nos. 4,876,187 and 5,011,769; Ogilvie et al., Proc. Natl. Acad. Sci. USA 85:8783-8798, 1987; Usman et al., J. Am. Chem. Soc. 109:7845-7854, 1987; Wu et al., Tetrahedron Lett. 29:4249-4252, 1988; Chaix et al., Nuc. Acids Res. 17:7381-7393, 1989; Wu et al., Nuc. Acids Res. 17:3501-3517, 1989; McBride and Caruthers, Tetiahedron Lett. 24:245-248, 1983; Sinha et al., Tetrahedron Lett. 24:5843-5846, 1983; Sinha et al., Nuc. Acids Res. 12:4539-4557, 1984; and Gasparutto et al., Nuc. Acids Res. 20:5159-5166, 1992.

The probes of the preferred embodiment are based on the cfb genes encoding CAMP and the sip gene encoding the surface immunogenic protein of GBS.

More particularly, preferred embodiments of the present invention include the probes identified herein as SEQ ID NOs: 5 and 10. The probes of SEQ ID NOs: 5 and 10 are double labeled with a fluorophore at the 5′ and a quencher at the 3′, so when the probe is intact the flourophore does not emit a detectable fluorescent signal (TaqMan®Probe, IDT, Coralville, Iowa). During PCR extension, the probes hybridized to the amplicons are cleaved by the 5′-3′ exonuclease activity of Taq polymerase, resulting in release of the 5′ fluorophore from its quencher. The intensity of the fluorescence increases as more amplicons are synthesized.

Briefly, oligonucleotide synthesis is accomplished in cycles wherein each cycle extends the oligonucleotide by one nucleotide. Each cycle consists of four steps: (1) deprotecting the 5′-terminus of the nucleoside or oligonucleotide on the solid support, (2) coupling the next nucleoside phosphoramidite to the solid phase immobilized nucleotide, (3) capping the small percentage of the 5′-OH groups of the immobilized nucleotides which did not couple to the added phosphoramidite, and (4) oxidizing the oligonucleotide linkage to a phosphotriester linkage.

Detection Reactions

As noted above, a wide variety of cycling reactions for the detection of a desired target nucleic acid molecule may be readily performed according to the general steps set forth above (see also, U.S. Pat. Nos. 5,011,769 and 5,403,711).

In another embodiment, Cycle ProbeTechnology (CPT) can be used for detecting amplicons generated by any target amplification technology. For example CPT enzyme immunoassay (CPT-EIA) can be used for the detection of PCR amplicons. CPT allows rapid and accurate detection of PCR amplicons. CPT adds a second level of specificity which will prevent detection of non-specific amplicons and primer-dimers. The PCR-CPT method may also be used for mismatch gene detection. Other variations of this assay include ‘exponential’ cycling reactions such as described in U.S. Pat. No. 5,403,711 (see also U.S. Pat. No. 5,747,255).

A lateral flow device (strip or dipstick) as described in U.S. Pat. Nos. 4,855,240 and 4,703,017, for example, represents another embodiment used for detection in the GBS assay. Instead of detecting uncleaved CAMP or Sip probe on streptavidin coated wells (i.e., EIA format), the uncleaved probe is captured by streptavidin impregnated on a membrane (i.e., strip format). There are several advantages for using this format. There are no additional detection reagents required, less hands-on time, and a short detection time. Representative examples of further suitable assay formats including any of the above assays which are carried out on solid supports such as dipsticks, magnetic beads, and the like (see generally U.S. Pat. Nos. 5,639,428; 5,635,362; 5,578,270; 5,547,861; 5,514,785; 5,457,027; 5,399,500; 5,369,036; 5,260,025; 5,208,143; 5,204,061; 5,188,937; 5,166,054; 5,139,934; 5,135,847; 5,093,231; 5,073,340; 4,962,024; 4,920,046; 4,904,583; 4,874,710; 4,865,997; 4,861,728; 4,855,240; 4,847,194 and 6,130,098).

In another embodiment, CPT can be carried out using the exponential formats with two sets of nucleic acid probe molecules, eg. CAMP and Sip which are immobilized on solid support as described in U.S. Pat. No. 5,403,711. This would be advantageous since the assay can be carried out in a single container, the signal can be monitored over time and would result in a very rapid and sensitive assay.

In yet another embodiment, CPT-EIA can be used for detecting GBS by use of reverse transcriptase to transcribe cDNA from mRNA expressed by the GBS gene followed by Cycling Probe Technology (RT-CPT) as described in U.S. Pat. No. 5,403,711. The uncleaved probe specific for the cDNA can than be detected by EIA.

In the area of DNA diagnostics, automated platforms based on labeled synthetic oligonucleotides immobilized on silicon chips work by fluorescence detection and are capable of the parallel analysis of many samples and mutations. Methods used in preparing labeled, chemically activated nucleotide precursors for oligonucleotide synthesis is discussed and demonstrated by Ruth et al. Nucleic acid amplification methods such as PCR have become very important in genetic analysis and the detection of trace amounts of nucleic acid from pathogenic bacteria and viruses. Analysis of many PCR reactions by standard electrophoretic methods becomes tedious, time consuming and does not readily allow for rapid and automated data acquisition. PCR has been adapted for use with fluorescent molecules by incorporation of fluorescently labeled primers or nucleotides into the PCR product which is then directly detected or detected indirectly using secondary probes, the binding of which is detectable. Removal of unincorporated, labeled substrates is usually necessary and can be accomplished by filtration, electrophoretic gel purification or chromatographic methods. However, the large amount of sample handling required by these analytical techniques make these purification methods labor intensive, not quantitative and they invariably leads to serious contamination problems. Affinity capture of PCR products by strepavidin coated beads or micro titer wells requires incorporation of biotin labels in addition to the fluorophores and still involves transfer steps that can lead to contamination. Instrumentation utilizing both gel electrophoresis and laser excitation optics represents an improvement in data acquisition but cannot handle large numbers of samples, retains the comparatively prolonged separation times characteristic of gels and still requires sample transfer.

Polynucleotide Amplification Techniques

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCRT, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Another method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™ bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Q beta Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]triphosphates in one strand of a restriction site (Walker et al., 1992, incorporated herein by reference in its entirety), may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods described in Great Britain Pat. Appl. No. 2 202 328, and in PCT Intl. Pat. Appl. Publ. No. PCT/US89/01025, each of which is incorporated herein by reference in its entirety, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of a second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, discloses a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H(RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Appl. No. WO 89/06700, incorporated herein by reference in its entirety, discloses a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “RACE” (Frohman, 1990), and “one-sided PCR” (Ohara, 1989) which are well-known to those of skill in the art.

Methods based on ligation of two (or more) oligonucleotides in the presence of a nucleic acid having the sequence of the resulting “di-oligonucleotide”, thereby amplifying the di-oligonucleotide (Wu and Dean, 1996, incorporated herein by reference in its entirety), may also be used in the amplification of DNA sequences of the present invention.

There is a need in the art for a method of detecting or measuring a target nucleic acid sequence from Group B Streptococcus that does not require multiple steps.

There is also a need in the art for a PCR process for detecting or measuring a target nucleic acid sequence from Group B Streptococcus that does not require multiple steps subsequent to the amplification process.

There is also a need in the art for a PCR process for detecting or measuring a target nucleic acid sequence from Group B Streptococcus that allows for concurrent amplification and detection of a target nucleic acid sequence in a sample.

The invention provides for a polymerase chain reaction process wherein amplification and detection of a target nucleic acid sequence from Group B Streptococcus occur concurrently (i.e. real time detection). The invention also provides for a polymerase chain reaction process wherein amplification of a target nucleic acid sequence occurs prior to detection of the target nucleic acid sequence (i.e. end point detection).

In another preferred embodiment, the nucleic acid polymerase is a DNA polymerase.

In another preferred embodiment, the nucleic acid polymerase is selected from the group consisting of Taq polymerase.

The invention also provides a kit for generating a signal indicative of the presence of a target nucleic acid sequence in a sample comprising a nucleic acid polymerase, a primer, a probe and a suitable buffer. In a preferred embodiment, the invention also provides a kit for generating a signal indicative of the presence of a target nucleic acid sequence from Group B Streptococcus in a sample comprising one or more nucleic acid polymerases, primers and probes and a suitable buffer. In a preferred embodiment, the target nucleic acid sequences are the cfb gene encoding for CAMP, and the Sip gene encoding for the surface immunogenic protein of GBS.

In another preferred embodiment the kit further comprises a labeled nucleic acid complementary to the target nucleic acid sequence.

Further features and advantages of the invention are as follows. The claimed invention provides a method of generating a signal to detect and/or measure a GBS target nucleic acid wherein the generation of a signal is an indication of the presence of a GBS target nucleic acid in a sample. The claimed invention also provides a PCR based method for detecting and/or measuring a target nucleic acid comprising generating a signal as an indication of the presence of a target nucleic acid. The claimed invention allows for simultaneous amplification and detection and/or measurement of a target nucleic acid sequence.

The practice of the present invention will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.).

Production of a Nucleic Acid

The invention provides for nucleic acids to be detected and or measured, and for amplification of a target nucleic acid sequence for identification of genes found in Group B Streptococcus.

Nucleic Acids Comprising Genomic DNA

Nucleic acid sequences of the invention are amplified from genomic DNA. Genomic DNA is isolated from tissues or cells collected from swabs according to the following method.

To facilitate detection of a gene from a particular tissue, the tissue is isolated free from surrounding normal tissues. To isolate genomic DNA from mammalian tissue, the tissue may be minced and frozen in liquid nitrogen. Frozen tissue may be ground into a fine powder with a prechilled mortar and pestle, and suspended in digestion buffer (100 mM NaCl, 10 mM Tris-HCl, pH 8.0, 25 mM EDTA, pH 8.0, 0.5% (w/v) SDS, 0.1 mg/ml proteinase K) at 1.2 ml digestion buffer per 100 mg of tissue. To isolate genomic DNA from mammalian tissue culture cells, cells are pelleted by centrifugation for 5 min at 500×g, resuspended in 1-10 ml ice-cold PBS, repelleted for 5 min at 500×g and resuspended in 1 volume of digestion buffer.

Samples in digestion buffer are incubated (with shaking) for 12-18 hours at 50° C. and then extracted with an equal volume of phenol/chloroform/isoamyl alcohol. If the phases are not resolved following a centrifugation step (10 min at 1700×g), another volume of digestion buffer (without proteinase K) is added and the centrifugation step is repeated. If a thick white material is evident at the interface of the two phases, the organic extraction step is repeated. Following extraction the upper, aqueous layer is transferred to a new tube to which will be added ½ volume of 7.5M ammonium acetate and 2 volumes of 100% ethanol. The nucleic acid is pelleted by centrifugation for 2 min at 1700×g, washed with 70% ethanol, air dried and resuspended in TE buffer (10 mM Tris-HCl, pH 8.0, 1 mM EDTA, pH 8.0) at 1 mg/ml. Residual RNA is removed by incubating the sample for 1 hour at 37° C. in the presence of 0.1% SDS and 1 μg/ml DNase-free RNase, and repeating the extraction and ethanol precipitation steps. The yield of genomic DNA, according to this method is expected to be approximately 2 mg DNA/1 g cells or tissue (Ausubel et al., supra). Genomic DNA isolated according to this method can be used for PCR analysis, according to the invention.

Polymerase Chain Reaction (PCR)

Nucleic acids of the invention may be amplified from genomic DNA or other natural sources by the polymerase chain reaction (PCR). PCR methods are well-known to those skilled in the art.

PCR provides a method for rapidly amplifying a particular DNA sequence by using multiple cycles of DNA replication catalyzed by a thermostable, DNA-dependent DNA polymerase to amplify the target sequence of interest. PCR requires the presence of a target nucleic acid sequence to be amplified, two single stranded oligonucleotide primers flanking the sequence to be amplified, a DNA polymerase, deoxyribonucleoside triphosphates, a buffer and salts.

PCR, is performed as described in Mullis and Faloona, 1987, Methods Enzymol., 155: 335, herein incorporated by reference.

The polymerase chain reaction (PCR) technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment whose termini are defined by the 5′ ends of the primers. PCR is reported to be capable of producing a selective enrichment of a specific DNA sequence by a factor of 10⁹. The PCR method is also described in Saiki et al., 1985, Science 230:1350.

PCR is performed using template DNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of a suitable buffer, 0.4 μl of 1.25 μM dNTP, 2.5 units of Taq DNA polymerase (Stratagene) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is generally used. Initial denaturation of the template molecules normally occurs at between 92° C. and 99° C. for 4 minutes, followed by 20-40 cycles consisting of denaturation (94°-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

In a particular embodiment of the present invention, the PCR procedure may be a real-time PCR procedure. Moreover, the PCR procedure employed may use the materials and methodology outlined in U.S. Pat. No. 6,130,098, incorporated herein by reference in its entirety.

Detection methods generally employed in standard PCR techniques use a labeled probe with the amplified DNA in a hybridization assay. Preferably, the probe is labeled, e.g., with ³²P, biotin, horseradish peroxidase (HRP), etc., to allow for detection of hybridization.

In a particular embodiment of the present invention, the probe utilized (SEQ ID NOs: 5 and/or 10), recognizes the sequence amplified between the primers, eg. the amplicons of the cfb gene comprised of the 123 base pair sequence of SEQ ID NO:4, allowing real-time detection by using fluorescence measurements. A further embodiment of the present invention includes a pair of GBS specific PCR amplification primers (SEQ ID NOs 6 and 7) specific for a portion of the sip gene (SEQ ID NO: 8) between positions 778 and 857 (SEQ ID NO: 9). Further included is a probe (SEQ ID NO: 10) recognizing the amplicons allowing real-time detection by using fluorescent measurement.

Other means of detection include the use of fragment length polymorphism (PCR FLP), hybridization to allele-specific oligonucleotide (ASO) probes (Saiki et al., 1986, Nature 324:163), or direct sequencing via the dideoxy method (using amplified DNA rather than cloned DNA). The standard PCR technique operates (essentially) by replicating a DNA sequence positioned between two primers, providing as the major product of the reaction a DNA sequence of discrete length terminating with the primer at the 5′ end of each strand. Thus, insertions and deletions between the primers result in product sequences of different lengths, which can be detected by sizing the product in PCR-FLP. In an example of ASO hybridization, the amplified DNA is fixed to a nylon filter (by, for example, UV irradiation) in a series of “dot blots”, then allowed to hybridize with an oligonucleotide probe labeled with HRP under stringent conditions. After washing, terramethylbenzidine (TMB) and hydrogen peroxide are added: HRP oxidizes the hydrogen peroxide, which in turn oxidizes the TMB to a blue precipitate, indicating a hybridized probe.

Oligonucleotide Design for Real-Time PCR Assays

There are several different approaches to real-time PCR. SYBR green detection is utilized with real time PCR because multiple reactions can be set-up rapidly and inexpensively using standard oligonucleotides. Real-time PCR relies on the fluorescent quantification of PCR product during each cycle of amplification. Specific detection systems, such as molecular beacons and Taqman assays rely on the synthesis of a fluorescently labeled detection oligonucleotide. These specific assays have the advantage of specificity, but the disadvantage of added expense and a delay in obtaining the fluorescently labeled detection oligonucleotides. Assay of PCR product through the use of the fluorescent dye SYBR green allows the reaction to be based on standard oligonucleotides. Because SYBR green will detect any PCR product, including non-specific products and primer-dimers, careful oligonucleotide design for the reaction is required.

Primers should be designed, if possible, within 1 kb of the polyadenylation site. Amplicons of 100-200 bp are ideal for real time applications. It is advantageous to design the primers to have the same melting temperature so that PCR with different primer sets can be performed in the same run. Primers that are 20-mers with 55% GC content and a single 3′-G or C can be used. Candidate primers are tested for specificity by BLAST and for folding and self annealing using standard DNA analysis software. Primer pairs are first tested for specificity and absence of primer-dimer formation (low molecular weight products) by PCR followed by gel electrophoresis. Designing each primer pair takes about one hour.

Real Time PCR

Real-time PCR requires a specialized thermocycler with fluorescent detection. A variety of commercial instruments are available. The ABI Prism 7700 allows assays to be performed in 96 well plate format. Good PCR technique is required to avoid contamination of subsequent reactions. This includes isolating PCR products and plasmids from RNA preparation and reaction setup. A dedicated bench for RNA isolation and PCR reaction set-up and dedicated pipettors should be maintained. Aerosol resistant pipette tips are used.

Commercial kits for SYBR green based PCR reactions are available from Applied Biosystems and perform reliably (SYBR Green PCR Core Reagents, P/N 4304886; SYBR Green PCR Master Mix, P/N 4309155).

“Hot start” taq polymeraase may be used. Platinum Taq, (Life Technologies), and Amplitaq gold, (Applied Biosystems), both perform well. The 10× SYBR Green I may be prepared by diluting 10 μl of the stock 10,000× concentrate (Cat# S-7563, Molecular Probes, Eugene, Oreg.) into 10 ml Tris-HCl, pH 8.0, and is stored in 0.5 ml aliquots at −20° C.

15 μl of the master mix are aliquoted into 0.2-mL MicroAmp optical tubes (P/N N801-0933, Applied Biosystems). Alternatively, a 96-well optical reaction plate (P/N 4306737, Applied Biosystems) can be used. Five μl of the first strand cDNA is then added to the tube and the solution is mixed by repeat pipetting. This achieves a final concentration reaction containing 20 mM Tris-, 50 mM KCl, 3 mM MgCl₂, 0.5× SYBR Green 1,200 μM dNTPs, 200 μM each of forward and reverse primers, approximately 500 pg first strand cDNA, and 0.5 units Taq polymerase.

The reaction tubes are covered with MicroAmp optical caps (P/N N801-0935, Applied Biosystems) using a cap installing tool (P/N N801-0438, Applied Biosystems). The contents are collected to the bottom of the tube by brief centrifugation in a Sorvall RT-6000B benchtop centrifuge fitted with a microplate carrier (PN 11093, Sorvall). The tubes are then placed in the ABI 7700 thermocycler and incubated at 95° C. for 2 minutes (10 minutes if using Amplitaq gold) to activate the enzyme and denature the DNA template. Forty cycles of PCR amplification are then performed as follows: Denature 95° C. for 15 seconds, Anneal 55° C. for 20 seconds, Extend 72° C. for 30 seconds.

This protocol works well for amplicons up to 500 base pairs and for amplicons up to about 150 base pairs in the instance of real time PCR. For longer amplicons, the extension step should be adjusted accordingly (approximately 1 minute per kb). Either the FAM or the SYBR channel can be used for fluorescence detection of SYBR Green I. Fluorescent emission values are collected every 7 seconds during the extension step. Data are analyzed using Sequence Detector version 1.7 software (Applied Biosystems). In order to obtain the threshold cycle (C_{T}) values, the threshold is set in the linear range of a semi-log amplification plot of ΔRn against cycle number. This ensures that the C_{T} is within the log phase of the amplification. Here the ΔRn is the fluorescence emission value minus baseline fluorescence value. When the PCR is at 100% efficiency, the C_{T} decreases by 1 cycle as the concentration of DNA template doubles.

In order to confirm that the correct amplicon is made, the amplified products are analyzed by agarose gel electrophoresis and visualized by ethidium bromide staining. A good reaction yields a single band of the expected size and has no smearing or primer-dimer formation.

To generate a standard curve for each primer pair, 10-fold serial dilutions are made from a plasmid with known number of copies of the gene. The C_{T} of each dilution is determined, and is plotted against the log value of the copy number. Amplification efficiency of each primer pair is obtained by the slope of regression. A 100% efficient PCR has a slope of −3.32. The number of copies in the samples is extrapolated by its C_{T} value using the respective standard curve.

Accordingly, the present invention resides in part in a process for amplifying two specific nucleic acid sequences present in a nucleic acid or mixture thereof, using two pairs of specific primers for polymerization, and two specific probes for detecting the amplified sequences. Further, the present invention provides an important advantage in allowing quick detection of the presence of the GBS pathogen so that appropriate medical intervention is available to the infection patient(s) more quickly.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Primer and Probe Design

The sequences of cfb and sip genes are obtained from GenBank. The primers and probes were designed with the aid of Primer Express 1.0 (PE Applied Biosystem). The possible homologies of the primers and probes with other none GBS genes were checked using NCBI Blast program and Megaline (DNA Star Lasergene).

CAMP based GBS-specific primers:
Forward primer:

(SEQ ID NO:1)

5′ GATGTATCTATCTGGAACTCTAGTG 3′;
Reverse primer:

(SEQ ID NO:2)

5′ GGCTTGATTATTACTATTTACATGATTTACCA 3′;
Probe:

(SEQ ID NO:5)

5′ F-AGAAGTACATGCTGATCAAGTGACAACTCCACA-Q 3′.
Sip based GBS-specific primers:
Forward primer:

(SEQ ID NO:6)

5′ GTGCATCACCAGAGCATGTAT 3′;
Reverse primer”

(SEQ ID NO:7)

5′ CGCTTGTAACTTACTGTCTGTAGCTG 3′;
Probe:

(SEQ ID NO:10)

5′ F-AGCTCCAGCAGTTCCTGTGACTACGACTT-Q 3′.

The specificity of the primers and probes was tested with real-time PCR (Taqman assay) using genomic DNAs isolated from the following organisms (listed in Table 1): nine GBS serotypes (serotype Ia, Ib, Ic, II, III, IV, V, VI and VII; American Type Culture Collection and National Center for Streptococcus, Canada); 10 clinical GBS isolates; 60 clinical samples; a wide variety of gram-positive and gram-negative bacterial strains as well as two yeast strains and HSV type 1 and 2.

Assay procedure. A typical PCR was conducted with the GBS specific probes and primers of the invention under the following conditions:

20 mM Tris-HCl, pH 8.4

50 mM KCl

4 mM MgCl₂

0.2 mM dNTPs

400 μM primers (SEQ ID NOs:1, 2, 4, 5)

200 μM probes (SEQ ID NOs:3, 6)

10 fg to 1 ng DNA

1-2 U of Taq polymerase (0.125 to 0.5 U/μl)

Total volume is 15 μl and the reaction is carried out in a LightCycler with: 25 sec denaturing at 94° C.; followed by 50 cycles of 94° C. for 3 sec., and 60° C. for 20 sec.

Results. Both sets of primers and probes recognized all the nine GBS serotypes, the 10 clinical isolates, and the clinical samples, which are GBS positive by culturing method. There are no cross-reactivities with any of the other pathogens.

### Example 2

### On-Chip PCR

The described primers and probes of the present invention can also be used for on-chip PCR. The on-chip PCRs were performed using the HandyLab prototype PCR machine (U.S. Pat. No. 6,130,098, incorporated herein by reference in its entirety). The specificity of the probes and primers were demonstrated by mixing GBS genomic DNA (20 to 200 copies) with DNA isolated from Streptococcus pyogenes (Group A strep or GAS) and Streptococcus pneumoniae (SP) (2000 to 20,000 copies). The presence of such contaminants did not detectably affect the assays results. Since biological samples are generally comprised of a plurality of microorganisms, the demonstration that such contaminants do not alter the specificity of the assay for detecting GBS-related nucleic acid sequences provides additional validation of the utility of the assay.

Assay Protocol

The described primers and probes were also used for on-chip PCR. A typical PCR was conducted with the GBS specific probes and primers of the invention under the following conditions:

20 mM Tris-HCl, pH 8.4

50 mM KCl

4 mM MgCl₂

0.2 mM dNTPs

400 μM primers (SEQ ID NOs: 1 and 2)

200 μM probes (SEQ ID NO:3)

10 fg to 1 ng DNA

0.125 to 0.5 U/μl of Taq polymerase

The on-chip PCRs were performed using HandyLab prototype PCR machine. All of the PCRs are performed with HL PCR chip in 1 □1 volume with 400 nM each CAMP primers and 200 nM CAMP probe. The specificity of the primers and probes were demonstrated by mixing GBS genomic DNA (20 to 200 copies) with DNA isolated from S. pyogenes (GAS) and S. Pneumoniae (SP) (2000 to 20,000 copies).

Results of the On-chip PCR Assay:

The results of the on-chip PCR assay revealed that an increase in fluorescence (ie. recognition of the GBS genomic sequence by the primers and probes described herein) was only observed over time when the PCR was conducted with GBS genomic DNA. However, when 25,000 fg of Streptococcus pyogenes (GAS) or Streptococcus pneumoniae (SP) genomic DNA was tested, there were no changes observed in fluorescence over time, indicating no amplification of DNA with the primers therefore no release of the labeled nucleodites from the probe of the present invention. Likewise, when genomic GBS DNA was mixed with genomic DNA from both GAS and SP, there was a significant increase over time in fluorescence, thus demonstrating the sensitivity of the reagents (primers and probes specific for GBS genes) utilized in this assay. Also, the negative controls (without DNA templates) demonstrated no change in fluorescence, once again demonstrating the specificity of the PCR reaction for genes of GBS using the probes and primers described within the present invention.

### Example 3

### Comparison of Two Primers Specific for the cfb Gene of GBS

Two primers were synthesized and compared for reactivity with GBS genomic DNA using an commercial real-time PCR machine, LightCycler. The sequence of one primer designated as CAMP1 is identified in SEQ ID NO: 2. The other primer designated as CAMP2 corresponds to SEQ ID NO: 11, which differs from CAMP1 at position 17, wherein the T from SEQ ID NO: 2 is replaced by a C. The detection limits of these two primers were tested with 8 serotypes of GBS. Both primers can detect GBS at 100 copies. However at 10 copies, the sensitivity of the two primers varies (reflected by Ct, critical cycle, number, in Table 1). Therefore both primers may be included in a PCR to ensure the sensitivity.

TABLE 1

| GBS | | |
|---|---|---|
| serotype | CAMP1(Ct) | CAMP2(Ct) |
| 1a | 31.85 | 34.69 |
| 1b | / | 35.91 |
| 1c | 33.38 | 35.22 |
| 2 | 33.47 | 34.63 |
| 3 | 33.62 | 34.79 |
| 5 | / | 35.76 |
| 6 | 31.52 | 31.54 |
| 7 | 34.05 | / |

Results: Table 1: Detection Limits of Different GBS Serotypes with CAMP1 and CAMP2.

Although there were differences in the detection limits of the two primers, (reflected by Ct, critical cycle, number), both primers exhibited significant sensitivity for the GBS nucleic acid target.

### Example 4

### Nucleic Acid Amplification Reaction on a Silicon-Based Substrate

This example describes a nucleic acid amplification reaction on a silicon-based substrate. The established DNA biochemistry steps for PCR occur within physiological conditions of ionic strength, temperature, and pH. Thus, the reaction chamber components have design limitations in that there must be compatibility with the DNA, enzymes and other reagents in solution.

To assess biocompatbility, components were added to a standard PCR reaction. The results indicated that crystalline silicon may not be the ideal material for biological compatibility. Given these results, it may be desirable to modify the surface of the micromachined silicon substrate with adsorbed surface agents, covalently bonded polymers, or a deposited silicon oxide layer.

To form a biologically compatible heating element, the present inventors began by coating a standard silicon wafer with a 0.5 μm layer of silicon dioxide. Next, a 0.3 μm deep, 500 μm wide channel was etched into the silicon oxide and gold or aluminum was deposited (0.3 μm thick). This inlay process results in a relatively planar surface and provides a base for deposition of a water-impermeable layer. The impermeable layer is made by a sequence of three plasma enhanced vapor depositions: silicon oxide (SiOₓ), silicon nitride (SiN_{y}), and silicon oxide (SiOₓ). Since the materials are deposited from the vapor phase the precise stoichiometries are not known. A thin metal heater design was used for this device rather than the doped-silicon resistive heaters previously demonstrated for micromachined PCR reaction chambers, since the narrow metal inlay allows viewing of the liquid sample through a transparent underlying substrate, such as glass or quartz. Also, the use of several independent heating elements permits a small number to operate as highly accurate resistive temperature sensors, while the majority of elements are functioning as heaters.

A device fabricated with metal resistive heaters and oxide/nitride/oxide coating was tested for biological compatibility and temperature control by using PCR amplification of a known DNA template sample. The reaction was carried out on the planar device using twenty microliters of PCR reaction mix covered with mineral oil to prevent evaporation. The reaction mixture was cycled through a standard 35-cycle PCR temperature cycling regime using the integral temperature sensors linked to a programmable controller. Since the reaction volume was significantly larger than intended for the original heater design, a polypropylene ring was cemented to the heater surface to serve as a sample containment chamber. In all test cases, the presence of amplified reaction products indicated that the silicon dioxide surface and the heater design did not inhibit the reaction. Parallel amplification experiments performed on a commercial PCR thermocycler gave similar results. A series of PCR compatibility tests indicated that the reaction on the device is very sensitive to controller settings and to the final surface material in contact with the sample.

From the above it should be evident that the present invention can be adapted for high-volume projects, such as genotyping. The microdroplet transport avoids the current inefficiencies in liquid handling and mixing of reagents. Moreover, the devices are not limited by the nature of the reactions, including biological reactions.

### Example 5

### Capture of GBS DNA from a Heterogeneous Sample

The primer and probes of the present invention can also be used for PCR after capturing GBS DNA with GBS, streptococcus, or bacteria specific oligonucleotides. This will allow specific capture or enhance GBS or bacteria DNA in a heterogeneous sample, such as a vaginal swab sample. Thus, this optional step may be used following DNA extraction to further increase the sensitivity of the present assay when testing a biological sample, which are generally heterogeneous in nature. A vaginal swab sample, for example, typically comprises such a heterogenous population of microorganisms.

Assay Protocol: A 25 base GBS CAMP gene specific oligonucleotide (ATGGGATTTGGGATAACTAAGCTAG) (SEQ ID NO: 12) was synthesized with Biotin labeled at the 5′ end. 500 pmol of the biotin labeled oligonucleotide was incubated with MPG Streptavidin magnetic beads (from CPG. Pre-washed with 2× binding buffer, 2M KCl, 10 mM Tris pH 7.5) in 100 □l of 0.5× binding buffer at room temperature for 5 min. The unbound oligos were removed and the beads were washed once with 100 ml washing buffer (2M NaCl, 10 mM Tris pH 7.5). 10 □l of GBS genomic DNA (1 ng) were denatured at 95° C. for 2 min and then placed on ice. The capture of the GBS genomic DNA was performed in 20 □l hybridization buffer (0.5 M NaCl, 10 mM Tris pH 7.5) containing oligo coated MPG beads. The mixture was incubated at room temperature for 5 min and washed once with 100 □l washing buffer. The release of the captured GBS DNA was carried out by incubating the magnetic beads in 5 □l dH₂O at 70° C. for 3 minutes and repeated once with another 5 ml dH₂O. One □l of the released DNA was used for PCR with CAMP specific primers (SEQ ID NOs: 1 and 2) and probes (SEQ ID NO: 3). PCR was performed in a LightCycler (Roche) with captured GBS genomic DNA as template.

Results

As shown in FIG. 3, the GBS CAMP gene specific oligonucleotide coated onto beads was able to specifically capture the GBS genomic DNA, whereas the negative control DNA was not captured to any significant degree.

### Example 6

### Construction of Nucleic Acid Probes

Nucleic acid molecules can be synthesized utilizing standard chemistries on automated, solid-phase synthesizers such as PerSeptive Biosystems Expedite DNA synthesizer (Boston, Mass.), PE Applied Biosystems, Inc.'s Model 391 DNA Synthesizer (PCR-MATE EP) or PE Applied Biosystems, Inc.'s Model 394 DNA/RNA Synthesizer (Foster City, Calif.). Preferably, PerSeptive Biosystems Expedite DNA synthesizer is used and the manufacturer's modified protocol for making oligonucleotides is carried out.

Reagents for synthesis of oligonucleotides are commercially available from a variety of sources including synthesizer manufacturers such as PerSeptive Biosystems, PE Applied Biosystems Inc., Glen Research (Sterling, Va.) and Biogenex. For DNA and RNA synthesis, the preferred fluorescein amidite, phosphoramidites of deoxy- and ribo-nucleosides, 2′-O-methyl and reagents, such as activator, Cap A, Cap B, oxidizer, and trityl deblocking reagent are available from PerSeptive Biosystems. Biotin-TEG-phosphoroamidite and Biotin-TEG-CPG are available from Glen Research. Ammonium hydroxide (28%) used for the deprotection of oligonucleotides is purchased from Aldrich. 1 M Tetrabutylammonium fluoride (TBAF) used for removing the 2′-O-tert-butyldimethylsilyl group is purchased from Aldrich and used after drying over molecular sieves for 24 hours. All buffers are prepared from autoclaved water and filtered through 0.2 μm filter.

The following procedure is used for preparing biotinylated and/or fluoresceinated oligonucleotides. Biotin-TEG-CPG (1 μmol) is packed into a synthesis column. Nucleoside phosphoramidites are then linked to make the defined nucleic acid sequence using PerSeptive Biosystem's modified protocol for making oligonucleotides. Fluorescein-amidite is dissolved in acetonitrile to a final concentration of 0.1 M. The fluorescein amidite is loaded on the synthesizer and added to the 5′-end of the oligonucleotide. Alternatively, phosphoramidite containing thio-linker is added at the 5′-terminal of the chimeric probe using the modified protocol. After the deprotection step described below, the probe is purified by reverse phase HPLC using Millipore's R-2 resin which retains the trityl containing oligonucleotide. In order to generate free reactive thio-group, the HPLC purified probe is treated with silver nitrate for 90 minutes at room temperature followed by neutralization of silver nitrate with dithiotheritol (DTT). The fluorescein-supernatant is then added to the free thio-group of the probe and then purified either by HPLC or by electrophoresis as described below.

After the synthesis of the oligonucleotide sequence, the resin bound oligonucleotide is treated initially with 25% ethanol-ammonium hydroxide (4 ml) at room temperature for 1 hour and subsequently at 55° C. for 16 hours in a closed tube. The tube is cooled, supernatant removed and concentrated to dryness in order to remove ammonia. The residue is dissolved in 1 ml of water and filtered through a 0.2 μm filter. The OD₂₆₀ is determined and an aliquot of approximately 2 OD₂₆₀. units is injected into the R-2 column of Biocad's HPLC to obtain a base line on the chromatogram for the tert-butyldimethylsilyl groups of the chimeric probe.

The remaining probe solution is lyophilized by centrifugal vacuum evaporator (Labconco) in a 1.5 ml microcentrifuge tube. The resulting oligonucleotide residue is deprotected with 1.0 M TBAF for 24 hours. To determine the extent of desilylation which has taken place, an aliquot of the TBAF reaction mixture is injected into the HPLC (R-2 column) using a linear gradient of 0 to 60% acetonitrile in 50 mM triethylammonium acetate (TEAA), pH 6.5. If only a partial desilylation has occurred, the TBAF reaction mixture is allowed to proceed for an additional 12 to 16 hours for complete removal of the protecting groups. The TBAF reaction mixture is quenched with 100 mM NaOAc, pH 5.5 and evaporated to dryness. The crude oligonucleotide product is desalted on a P-6 column (2 cm.times. 10 cm, Bio-Rad), the fractions are concentrated to approximately 1 ml and the concentration measured at OD₂₆₀.

The crude oligonucleotide is purified by polyacrylamide gel electrophoresis (PAGE) using 20% polyacrylamide-7 M urea. The running gel buffer is 1×TBE (Tris-Borate-ethylenediamine tetraacetic acid (EDTA), pH 8.3) and the electrophoresis is carried out at 50 mA current for 3.5 to 4 hours. The oligonucleotide band is visualized with UV light, excised, placed in a 15 ml plastic conical tube and extracted by crushing and soaking the gel in 5 ml of 50 mM NaOAc (pH 5.5) for approximately 12 hours. The tubes are then centrifuged at 3000 RPM and the supernatant carefully removed with a Pasteur pipette. The gel is rinsed with 2 ml of the extraction buffer to remove any residual product. The combined extract is concentrated to a volume of approximately 1 ml and desalted on a P-6 column. The fractions containing the probe are pooled and concentrated to a final volume of approximately 2 ml. The analytical purity of oligonucleotides is checked by labeling the 5′-end of oligonucleotide with γ³²P]-ATP and T4-polynucleotide kinase and then running the labeled oligonucleotide on PAGE. OD₂₆₀ is measured using Hewlett Packard's 845×UV spectrophotometer. The oligonucleotide solution is filtered through a 0.2 μm filter and stored at −20° C.

### Example 7

### GBS Test Kit: Format Reagent and Kit Composition

The following is one representative example of a kit for detecting the CAMP and, optionally, Sip genes from GBS. This kit allows for rapid detection of GBS by detecting the CAMP and, optionally, Sip genes using a real time PCR assay.

The Rapid GBS Test Kit (48 tests) is composed of the following items:

GBS Lysis Reagent (2)

GBS Cycle Reagent (48)

Wash Buffer (1×50 mL)

GBS Lysis Reconstitution Buffer (1×3 mL)

Detection Substrate Reagent (1×12 mL)

Cycle Reconstitution Buffer (1×6 mL)

Detection Stop Reagent (1×5.5 mL)

GBS Cycle Stop Reagent (1× Transfer Pipette (50)

50 μL Dropstir (75)

50 μL Dropstir (75)

200 μL Dropstir (50)

200 μL Dropstir (50)

The following describes the composition, reagents and materials that form part of the kit:

GBS Lysis Reconstitution Buffer: Water and 20 ppm ProClin 300.™. (Sigma).

GBS Lysis Reagent (lyopholized): TES, Triton X-100™Trehalose (Sigma),

Achromopeptidase (Wako) and EGTA

Cycle Reconstitution Buffer: 4 mM MgCl.sub.2 and 20 ppm ProClin 300™.

GBS Cycle Reagent (lyopholized): Trehalose, Polyvinylpyrrolidone, TES, Triton X-100™ spermine, GBS probe, RNase H and bovine serum albumin.

GBS Cycle Stop Reagent: Buffered Salt Solution (DAKO) containing anti-fluorescein antibody conjugated with horse radish peroxidase ( 1/1000 final dilution).

Streptavidin Coated Microwell (Boehringer).

Wash Buffer: 137 mM NaCl, 2.7 mM KCl, 1.8 mM KH₂ PO.sub.4, 10.1 mM Na₂ HPO₄, 0.5% Tween 20 and 20 ppm ProClin 300.

Detection Substrate Reagent: Tetramethylbenzidine (Sigma) and H₂O₂.

Detection Stop Reagent: 0.75 mM Tris and 1.5% Sodium dodecyl sulfate.

The procedure for carrying out the assay for detecting the CAMP and Sip genes from the crude lysates of Group B Streptococcus is as follows:

A. Reconstitution of GBS Lysis Reagent

1. To a vial of GBS Lysis Reagent pipette 1.5 mL of GBS Lysis Reconstitution Buffer.

2. Swirl to dissolve.

3. Let sit at room temperature for 2 to 3 minutes before use.

4. Once reconstituted a vial of GBS Lysis Reagent can be used for 2 weeks when stored at 2-8° C.

B. Reconstitution of GBS Cycle Reagent

1. The reconstitution should be performed during the incubation steps of Specimen Preparation. 2. To a vial of GBS Cycle Reagent add 2 drops of the Cycle Reconstitution Buffer. 3. Swirl to dissolve. 4. This is a single use reagent. This reagent must be used within 30 minutes of reconstitution.

C. Sample Preparation

1. Using a 50 μL Dropstir add one drop of the reconstituted GBS Lysis Reagent to each 1.5 mL microcentrifuge tube. (one tube per sample)

2. Add 1 μL loop of growth from an 18 to 24 hour culture on a tryptic soy agar plate containing 5% sheep blood. Mix well to completely suspend cell growth.

3. Place at 55° C. for 20 minutes.

4. Place at 95° C. for 5 minutes.

D. Cycling Probe Technology

1. Transfer tubes with lysate to 55° C.

2. Using a 50 μL Dropstir add one drop of the reconstituted GBS Cycle Reagent to each tube.

3. Incubate at 55° C. for 25 minutes.

4. Add 3 drops of GBS Cycle Stop Reagent, with tubes at 55° C.

E. Detection

1. Place the necessary number of Streptavidin Coated Microwells (one Microwell per sample) into the Microwell frame.

2. Transfer the entire cycle reaction to Streptavidin Coated Microwell using a transfer pipette.

3. Incubate at room temperature for 10 minutes.

4. Invert Streptavidin Coated Microwell to discard liquid.

5. Fill each Streptavidin Coated Microwell completely with Wash Buffer.

6. Invert Streptavidin Coated Microwell to discard liquid.

7. Tap each Streptavidin Coated Microwell 5 times on dry paper towel.

8. Repeat steps 5-7.

9. Using a 200 μL Dropstir add one drop of Detection Substrate Reagent to each of the Streptavidin Coated Microwells.

10. Place at room temperature for 5 minutes.

11. Add 4 drops of Detection Stop Reagent to each Streptavidin Coated Microwell. 12. Mix for 10 seconds.

13. Incubate at room temperature for 3 minutes.

14. Within 30 minutes visually read and record the color zone or measure/record the OD₆₅₀.

### Example 8

### Microfluidic Genotyping Chip

The microfluidic genotyping chip will consist of three component layers. These layers will include the PC board which houses the circuitry needed to operate the on-chip heaters; a sensor chip which houses the heaters and temperature sensors that will be flip-chip bonded to the PC board; and the fluidic chip which houses the microchannel network that will be placed directly on the heater chip. The sensor chip will be fabricated by HandyLab at the University of Michigan Solid State Electronics Laboratory. Several different heater designs will be utilized in order to provide the best temperature control and uniformity, as well as high heating and cooling rates. The microfluidic channel will be sent to a licensed vender for production and will be fabricated using plastic injection molding technology. A commercially available cyclic-olefin copolymer (TOPAS) will be used to fabricate the microchannel network. Previous work at HandyLab has shown the effectiveness of this material for PCR. The printed circuit board will be designed by HandyLab and manufactured/assembled by licensed vendors. The final product will be assembled at HandyLab and will include an integrated composite of PC board, heater chip and microfluidic chip.

TABLE 2

| PATHOGEN | TYPE |
|---|---|
| Pseudomonas aeruginosa | Gram − Bacteria |
| Proteus mirabilis | Gram − Bacteria |
| Klebsiella oxytoca | Gram − Bacteria |
| Klebsiella pneumoniae | Gram − Bacteria |
| Escherichia coli (clinical isolate 1) | Gram − Bacteria |
| Escherichia coli (clinical isolate 2) | Gram − Bacteria |
| Acinetobacter baumannii | Gram − Bacteria |
| Serratia marcescens | Gram − Bacteria |
| Enterobacter aerogenes | Gram + Bacteria |
| Enterococcus faecium | Gram + Bacteria |
| Staphylococcus aureus (clinical isolate 1) | Gram + Bacteria |
| Staphylococcus aureus (clinical isolate 2) | Gram + Bacteria |
| Streptococcus pyogenes | Gram + Bacteria |
| Streptococcus viridans | Gram + Bacteria |
| Listeria monocytogenes | Gram + Bacteria |
| Enterococcus sps. | Gram + Bacteria |
| Candida glabrata | Yeast |
| Candida albicans | Yeast |
| Streptococcus Group C | Gram + Bacteria |
| Streptococcus Group G | Gram + Bacteria |
| Streptococcus Group F | Gram + Bacteria |
| Enterococcus faecalis | Gram + Bacteria |
| Streptococcus pneumoniae | Gram + Bacteria |
| Staphylococcus epidermidis (C-) | Gram + Bacteria |
| Gardenerella vaginalis | Gram + Bacteria |
| Micrococcus sps. | Gram + Bacteria |
| Haemophilus influenzae | Gram − Bacteria |
| Neisseria gonorrhoeae | Gram − Bacteria |
| Moraxella catarrahlis | Gram − Bacteria |
| Salmonella sps. | Gram − Bacteria |
| Chlamydia trachomatis | Gram − Bacteria |
| Peptostreptococcus productus | Gram + Bacteria |
| Peptostreptococcus anaerobius | Gram + Bacteria |
| Lactobacillus fermentum | Gram + Bacteria |
| Eubacterium lentum | Gram + Bacteria |
| Herpes Simplex Virus I (HSV I) | Virus |
| Herpes Simplex Virus II (HSV II) | Virus |

### Example 9

### Determination of Specificity

Vaginal/rectal swabs were placed in ˜2 ml of GBS enrichment broth. About 0.4-1 ml of the sample was obtained. The samples were split to half, one for HL testing and one for IDI testing. For IDI testing, the cells were spun down, and the samples were resuspended in their lysis buffer in a volume adjusted so that it would be proportional to what it should be if the entire 2 ml sample was used for the testing. Therefore there was no dilution of the sample. The remainder of the experiment was performed following the instructions provided by Cepheid together with the primers provided in the GBS detection kit commercially available as IDI Catalog No. IDI-2002-001. The kit contains a probe and primers. The target sequence of the Cepheid kit is the cfb gene, and a 154 base pair fragment of cfb gene is amplified. The probe provided in the kit is a molecular beacon.

For the comparison method, the cells were spun down and resuspended in 1 ml of standard HL sample collection solution. A sample (1 ml) was drawn into a 3 ml syringe. Pre-filtration was performed with a custom made syringe filter, which contains both 10 and 3 micron filters. The filtrate was collected in a clean microcentrifuge tube to which the following was added: 0.4 mg of protease K, 0.8 mg of pronase, 18 U of RNase A, 75 units of mutanolysin, and 10 μL of HL DNA capture microsphere (˜5% solid/v). The tube was inverted several times to mix the sample well. It was then incubated at 60° C. for 10 minutes. The DNA capture microsphere spun down in a microfuge at 14 krpm for 7 min. The supernatant was discarded, and the pellet was resuspended in 20 □l washing solution. The tube was then vortexed until the microspheres were evenly suspended.

The tube was then spun in a microfuge at 14 k rpm for 7 min. The supernatant was discarded, and the pellet was resuspended in 4 □l 20 mM NaOH. The tube was then vortexed until the microspheres were evenly suspended. Bound DNA was released at 85 degrees C. for 2 min. The microsphere was spun down at 14 krpm for 7 min. 3.5 □l of the released DNA was neutralized with 1.5 ul of 120 mM Tris pH 8.0. 1 □l of the released DNA was used in a 4 μl PCR procedure. The results are presented in Table 3.

TABLE 3

| | Inventive/ | Inventive | | | |
|---|---|---|---|---|---|
| | Comparison | Pos | Neg | Total | |
| Comparison | Pos | 13 | 0 | 13 | Clinical |
| | | | | | sensitivity 100% |
| | Neg | 9* | 72 | 81 | Clinical |
| | | | | | specificity 89% |
| | Total | 22 | 72 | 94 | pos predic value |
| | | | | | 59% |
| | | | | | neg predic value |
| | | | | | 100% |

Among the 9 HL's “false positive”, or Cepheid's “false negative” samples, 4 of them were GBS po [symbol] determined by culturing method.

### Example 10

### Determination of Specificity

Group B Streptococci were obtained from the sources indicated in Table 4. Purified genomic DNA was therefore used to determine the sensitivity of detection using the methods of the present invention. PCR assays were performed to detect and quantify the Group B Streptococci in accordance with the procedures described in Example 9. The results including detection limit are set forth in Table 4. DNA is normally calculated as ug or fg, etc. Since the genome size of GBS is known as about 2 fg, it is possible to calculate the genome copy number equivalent. For instance, if it is possible to detect 20 fg of GBS DNA in the sample by PCR, the amount may be expressed as 10 genome copy equavalents or 10 GBS cells actually detected.

TABLE 4

| | | Detection limit |
|---|---|---|
| GBS | | (genome copy |
| serotype | Source | number equivalent) |
| Ia | ATCC 12400 | 20 |
| Ib | NCS, blood | 10 |
| Ic | ATCC 27591 | 10 |
| II | ATCC 12973 | 20 |
| III | ATCC BAA- | 10 |
| | 22 | |
| III | ATCC 12403 | 10 |
| IV | ATCC 49446 | 10 |
| V | ATCC 700046 | 40 |
| V | ATCC 49447 | 15 |
| V | ATCC BAA- | 10 |
| | 611 | |
| VI | NCS, Placenta | 5 |
| VII | NCS, blood | 10 |
| VIII | Clinical | 10 |
| | Isolate | |
| ND | ATCC 12928 | 15 |
| ND | ATCC 13813 | 10 |

We claim:

1. A method for detecting the presence of Streptococcus agalactiae, comprising:
(a) contacting a nucleic acid sample suspected of being infected with Group B Streptococcus (GBS), with a pair of CAMP-based Group B Streptococcal (GBS)-specific primers, under conditions wherein GBS-related nucleic acids are amplified; and
(b) detecting the presence of GBS-related nucleic acids, wherein detection of GBS related nucleic acids in the sample is a positive indicator of Streptococcus agalactiae (group B streptococcus) infection.

2. A method for detecting the presence of Streptococcus agalactiae, comprising:
(a) hybridizing a sample obtained from a bacterial culture or a patient suspected of being infected with Group B Streptococcus with (i) a first pair of CAMP-based Group B Streptococcal (GBS)-specific primers, and (ii) a second pair of Sip-based GBS-specific primers, under conditions wherein GBS-related nucleic acids are amplified; and
(b) detecting the presence of GBS-related nucleic acids, wherein detection of GBS related nucleic acids in the sample is a positive indicator of Streptococcus agalactiae (group B streptococcus) infection.

3. The method of claims 1 or 2, wherein 150 or less nucleic acid base pairs are amplified.

4. The method of claims 1 or 2, wherein the pair of CAMP-based Group B Streptococcal (GBS)-specific primers are the nucleic acid sequences of SEQ ID NOs:1 and 2.

5. The method of claim 1, wherein the pair of CAMP-based Group B Streptococcal (GBS)-specific primers are the nucleic acid sequences of SEQ ID NOs:1 and 11.

6. The method of claim 2, wherein the second pair of Sip-based GBS-specific primers are the nucleic acid sequences of SEQ ID NOs:4 and 5.

7. The method of claim 1, wherein step (a) is conducted in the presence of labeled probes comprising SEQ ID NO:3.

8. The method of claim 1, wherein the nucleic acid sample is a biological sample obtained from a patient to be tested for the presence of GBS.

9. The method of claim 1 or claim 2, wherein step (a) in conducted in a volume of between 0.2-100 μl.

10. The method of claim 1 or claim 2, wherein the nucleic acid is extracted from a biological sample obtained from a patient suspected of being infected with GBS.

11. A method of amplifying a nucleic acid related to Group B Streptococcus (GBS), comprising:
(a) contacting a GBS-related target nucleic acid with a pair of CAMP-based Group B Streptococcal (GBS)-specific primers, and a CAMP-based Group B Streptococcal (GBS)-specific probe, under conditions wherein GBS-related nucleic acids are amplified; and
(b) detecting the amplified product.

12. The method of claim 11, further comprising contacting the GBS-related target nucleic acid with a pair of Sip-based GBS-specific primers.

13. The method of claim 11, wherein step (a) is conducted in the presence of labeled probes comprising SEQ ID NO:3.

14. The method of claim 11, wherein step (a) in conducted in a volume of between 0.2-100 μl.

15. An method for detecting the presence of Streptococcus agalactiae in a biological sample in vitro, comprising:
(a) releasing nucleic acids from said biological sample;
(b) performing PCR in a total volume of between 0.2-100 μl in the presence of a pair of primers comprising SEQ ID NOs:1 and 2, or SEQ ID NOs 1 and 11, under conditions wherein the presence of a Streptococcus agalactiae-related nucleic acid sequence results in an amplified and labeled PCR product; and
(c) detecting the presence of a labeled PCR product.

16. A method detecting a Group B Streptococcal (GBS) infection in a patient, comprising:
(a) obtaining a biological sample from the patient;
(b) releasing nucleic acids from said biological sample;
(c) performing PCR in a total volume of between 0.2-100 μl in the presence of a pair of primers comprising SEQ ID NOs:1 and 2, or SEQ ID NOs: 1 and 11, and labeled probes comprising SEQ ID NO:3, under conditions wherein the presence of a Streptococcus agalactiae-related nucleic acid sequence results in an amplified and labeled PCR product; and
(d) detecting the presence of a labeled PCR product.
